(19) United States
(12) Patent Application Publication
(10) Pub. No.: US 2025/0288372 A1
(43) Pub. Date: Sep. 18, 2025

(54) SURGICAL ROBOTIC SYSTEM

(71) Applicant: GLOBUS MEDICAL, INC., AUDUBON, PA (US)

(72) Inventors: Norbert Johnson, North Andover, MA (US); David Cleary, Dracut, MA (US); Danielle Reinhard, Lafayette Hill, PA (US); Daniel Gehriger, Lausanne (CH); Robert Stevens, North Chelmsford, MA (US); Saumya Shah, Newton, MA (US); Douglas Mandell, Wakefield, MA (US); Mark Weiman, Downingtown, PA (US); Jay Martin, Medford, NJ (US); David C. Paul, Phoenixville, PA (US)

(21) Appl. No.: 18/635,238

(22) Filed: Apr. 15, 2024

Related U.S. Application Data
(63) Continuation-in-part of application No. 18/603,494, filed on Mar. 13, 2024.

(51) Int. Cl. A61B 34/30; A61B 34/20; A61B 50/13; A61B 90/00; A61B 90/50

(52) U.S. Cl. CPC A61B 34/30; A61B 50/13; A61B 90/37; A61B 90/50; A61B 2034/2055; A61B 2034/2063; A61B 2090/3937

(57) ABSTRACT

Devices, systems, and methods for robot-assisted surgery. A surgical robotic system with integrated navigation and multiple surgical arms may assist a user with one or more surgical procedures. The base station may include a motorized propulsion and positioning system to transport the robotic system. The system may utilize a powered machine vision end effector, which couples to the surgical arm, to provide specialized motion to an instrument. A sterile drape assembly may maintain sterility and preserve electrical connectivity. Ultrasound tracking may be used for registration, patient tracking, or guided tracking of instruments, for example.

### CROSS-REFERENCE TO RELATED APPLICATION

This application is a continuation-in-part of U.S. patent application Ser. No. 18/603,494 filed on Mar. 13, 2024, which is incorporated by reference herein in its entirety for all purposes.

### FIELD OF THE INVENTION

The present disclosure relates to medical devices, and more particularly, robotic surgical systems and related devices and methods.

### BACKGROUND OF THE INVENTION

Computer-assisted technology may be used during surgery, for example, to improve accuracy, reduce surgical time, and lower potential radiation exposure. In general, navigation gives surgeons better visualization in minimally invasive procedures while surgical robotics assists with trajectory alignment and positioning of the implants. The combination of robotics and navigation enhanced computer-assisted technology as robotics automated the positioning of the navigation. In addition, a robotic arm may be used to precisely align and hold the desired trajectory for the surgeon during the procedure.

There are several limitations to current robotic navigation systems, however. For example, systems may be limited by: navigation fiddle factors, such as inaccurate registrations or poor line of sight by the robotic camera; issues with passive guidance, such as possible patient movement, inability to actively move the system during the procedure, and difficulty working with long surgical constructs; having only a single robotic arm limiting methodology to one surgical action at a time; surgeons or assistants lacking visibility of the monitor to oversee the procedure; and overall system movement being hindered intraoperatively and/or during transport.

Thus, there remains a need for improved systems and methods for robot-assisted surgeries with robotic navigation systems that act as a tool and true assistant to the surgeon throughout the surgical procedure with the flexibility and adaptability for various clinical applications and approaches.

### SUMMARY OF THE INVENTION

To meet this and other needs, devices, systems, and methods for robot-assisted surgeries are provided. A surgical robotic system with integrated navigation and multiple surgical arms may assist a user with one or more surgical procedures. End effectors may be attached to each surgical arm to engage instrumentation and perform the desired surgery. In addition to the surgical arms, the robotic system may also have peripheral arms to position the navigation camera and surgeon displays. The robotic system is collaborative such that motorized sub-systems may be controlled both by the system software and manually by the user. These collaborative sub-systems may include all robotic arms, base motion, and base lock-out/stabilization. The collaborative design enables easy integration into procedural workflows, for example, to install pedicle screws, interbody implants, or other surgical devices.

According to one embodiment, a multi-arm surgical robotic system includes a moveable base station, including an on-board computer, the base station having two front wheels and two rear wheels attached to a bottom tray, and a handle for directional control of the base station, wherein at least one of the wheels is powered by a motor, a display electronically coupled to the computer, a camera electronically coupled to the computer and configured to detect one or more tracking markers, and a pair of surgical arms electronically coupled to the computer and movable based on commands processed by the computer.

The multi-arm surgical robotic system may include one or more of the following features. The rear wheels may be steerable casters with motorized propulsion. The motor may be a stepper motor mounted to a top of the bottom tray. The powered wheel may include a splined shaft connected to an output shaft of a reduction gearbox for the motor. The splined shaft may connect to a central shaft, which engages miter gearing to transmit torque to the wheel. The powered wheel may include a manual override assembly including a release lever having a release shaft and a release fork, which disengages the splined shaft from the central shaft. Alternatively, the wheels may include powered omni-directional (mecanum) wheels. The handle may be coupled to a steering shaft connected to a steering sprocket in the base station. The steering sprocket may be coupled via chain to sprockets fitted to each of the drive wheels, thereby synchronizing steering control to both rear wheels. The base station may also include a stabilizer assembly including a stationary housing and an inner stabilizer shaft. The inner stabilizer shaft may be configured to protrude from the end of the stationary housing and contact the floor to stabilize the base station. The inner stabilizer shaft may define a helical groove configured to engage a ball bearing in the stationary housing to guide deployment of the stabilizer shaft. The helical groove may have a variable helix with a deployment portion and a stabilization portion having a lead lower than the deployment portion.

According to one embodiment, a surgical robotic system includes a moveable base station, including an on-board computer, a machine vision camera electronically coupled to the computer, a surgical arm electronically coupled to the computer and movable based on commands processed by the computer, and an end effector attachable to the surgical arm. The end effector has an end effector base and an instrument adaptor. The instrument adaptor is configured to hold an instrument. The end effector base and/or the instrument adaptor is powered to provide specialized motion to the instrument. The end effector includes one or more machine vision markings so that the machine vision camera is able to determine a precise location of the end effector in real-time.

The surgical robotic system may include one or more of the following features. The machine vision markings may include quick-response (QR) codes. A first machine vision marking may be located on the end effector base and a second machine vision marking may be located on the instrument adaptor. The end effector base may include a housing with an adaptor rail track, and the instrument adaptor may include an adaptor rail configured to slidably mate with the adaptor rail track. The end effector base may include a hall sensor array next to the adaptor rail track, and the instrument adaptor may include a magnet. The hall sensor array may include a linear pattern of hall sensors that detect the location of the instrument adaptor along the adaptor rail track. The end effector base may include a control board providing bi-directional communication to the surgical arm and instrument adaptor, and a base motor and gearhead for providing rotary motion to the instrument. The instrument adaptor may include a motor configured to provide rotational or oscillating motion to the instrument. The instrument adaptor may include a battery and wireless transmitter and/or receiver for providing wireless communication to the on-board computer.

According to one embodiment, a method of robotic navigation may include one or more of the following steps in any suitable order: (1) providing a multi-arm surgical robotic system comprising a pair of surgical arms, a display, and a machine vision camera supported on a single mobile cart, and a separate powered machine vision end effector having an end effector base, an instrument adaptor, and an instrument; (2) attaching the end effector base to one of the surgical arms of the multi-arm surgical robotic system; (3) inserting the instrument adaptor, top down, into the end effector base by sliding an adaptor rail of the instrument adaptor into an adaptor rail track of the end effector base; (4) attaching the instrument to the instrument adaptor with a quick connector; (5) positioning the multi-arm surgical robotic system near an operating room table; and (6) performing a surgical procedure with the assistance of the surgical arms. The slidable mating of the instrument adaptor with the end effector base may allow for guidance control and assistance control. For guidance control, the surgical arm moves to a linear trajectory and a user controls a depth of the instrument along the linear trajectory, and for assistance control, the instrument adaptor is securely attached to the end effector base for active movement by the surgical arm. When a drive button on the instrument adaptor is depressed, a signal may be sent to the control board to rotate a motor in the end effector base at a given speed, thereby rotating the instrument.

According to one embodiment, a surgical robotic system includes a moveable base station, including an on-board computer, a display electronically coupled to the computer, a camera electronically coupled to the computer and configured to detect one or more tracking markers, a surgical arm electronically coupled to the computer and movable based on commands processed by the computer, an end effector attachable to the surgical arm, and a sterile drape assembly attachable to the surgical arm. The sterile drape assembly includes a sterile drape and a connector cap attached to the sterile drape. The sterile drape assembly provides for power and data transmission from the surgical arm to the end effector.

The surgical robotic system may include one or more of the following features. The connector cap may house an electrical connector, which transmits power and data from the surgical arm to the end effector. The surgical arm may include an end effector interface having a mounting flange with a conducive pad and a ferrous target. The end effector may include a clamp with a mounting flange, conducive pad, and ferrous target. The electrical connector of the connector cap may include a printed circuit board with pogo pins and a magnet on each side. The magnets on the connector cap allow for a magnetic connection with the ferrous targets on the surgical arm and end effector, respectively. A first set of pogo pins on the connector cap may align with the conducive pad on the surgical arm and a second set of pogo pins on the connector cap may align with the conducive pad on the end effector, thereby establishing electrical connectivity from the surgical arm to the end effector. The connector cap may include an embossed edge to facilitate proper alignment when attaching the connector cap to the surgical arm.

According to one embodiment, a multi-arm surgical robotic system includes a moveable base station, including an on-board computer, a display electronically coupled to the computer, a camera electronically coupled to the computer and configured to detect one or more tracking markers, a pair of surgical arms electronically coupled to the computer and movable based on commands processed by the computer, and an ultrasound probe to register or track patient anatomy of a patient during a surgical procedure.

The surgical robotic system may include one or more of the following features. An automatic ultrasound registration process may be configured to be completed by the system. The automatic ultrasound registration may include a rough calibration by scanning the patient anatomy and a fine calibration for focused images. During the automatic ultrasound registration, frames of optical tracking data may be synchronized with ultrasound data. The ultrasound probe may be attached to one of the surgical arms and automatically controlled by the system to intelligently guide the ultrasound imaging. The ultrasound probe may include a flexible multi-part ultrasound sensor configured to adhere to the patient. The ultrasound probe may include multiple ultrasound sensors configured to mount to the skin of the patient to track movement of the patient anatomy. The multiple ultrasound sensors may include machine vision markings for optical tracking of the sensors.

According to one embodiment, a method by a surgical robot system may include one or more of the following steps in any suitable order: (1) providing a multi-arm surgical robotic system comprising a pair of surgical arms, a display, and a camera supported on a single mobile cart, and an ultrasound probe, which is integrated into the surgical arm, integrated into an end effector, or offered as a separate component; (2) positioning the multi-arm surgical robotic system near an operating room table; (3) scanning a patient with the ultrasound probe to register or track patient anatomy; and (4) performing a surgical procedure with the assistance of the surgical arms. The method may further include: (5) performing a rough calibration by automatically scanning the patient with the ultrasound probe, and once anatomy is identified, replacing the rough calibration with a fine calibration to optimize imaging; (6) one surgical arm may be dedicated to holding the ultrasound probe, and positioning the ultrasound probe over regions of interest to track any movement from the patient, while the other surgical arm performs any surgical tasks; and (7) attaching ultrasound sensors to the skin of the patient at a later time to track the patient's progress.

Also provided are kits including implants of varying types and sizes, instruments, and other components for performing the procedures.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 illustrates a surgical robotic system having two surgical arms in accordance with one embodiment;

FIGS. 2A-2B show the surgical robotic system of FIG. 1 in deployed and docked system configurations, respectively;

FIGS. 3A-3B show profile and front views, respectively, of the docked system according to one embodiment;

FIG. 4 shows the deployed surgical robotic system positioned next to an operating room table;

FIG. 5 shows a robot base with motorized propulsion, directional control, and stabilization according to one embodiment;

FIG. 6 shows a cross-sectional view of a motorized caster wheel providing motorized propulsion to the system according to one embodiment;

FIGS. 7A-7B show the motorized caster wheel with a manual release lever in disengaged and engaged positions, respectively;

FIGS. 8A-8B show a robot base with a user input handle and steering shaft coupled to a steering gear and drive chain assembly according to one embodiment;

FIG. 9 shows an alternative robot base with four omnidirectional drive units for propulsion and steering control according to one embodiment;

FIGS. 10A-10B show a drive unit for the omnidirectional wheel according to one embodiment;

FIG. 11 shows a suspension assembly for the omnidirectional drive unit according to one embodiment;

FIG. 12 shows a manual system for decoupling the omnidirectional wheel from the drive unit according to one embodiment;

FIGS. 13A-13B show a close-up view of the decoupling key configured to engage and disengage with the decoupling hub for coupling and decoupling of the wheel, respectively;

FIGS. 14A-14B show cross-sectional views of the decoupling assembly decoupled and coupled, respectively, according to one embodiment;

FIG. 15 shows an example of a self-locking selector knob to prevent inadvertent actuation of the decoupling mechanism;

FIGS. 16A-16B show one embodiment of a stabilizer assembly and stabilizer shaft configured to restrict all relative motion between the robot base and the floor;

FIGS. 17A-17B show an alternative embodiment of a stabilizer assembly in retracted and deployed positions, respectively, having a scissor linkage configured to guide deployment of the stabilizer assembly;

FIGS. 18A-18B show an alternative embodiment of a stabilizer assembly in retracted and deployed positions, respectively, having linked carriages configured to deploy a stabilization foot;

FIG. 19 shows an underside of the robot base including front and rear stabilizers connected via a chain drive system for synchronized deployment according to one embodiment;

FIGS. 20A-20B show one embodiment of an active machine vision end effector configured for navigated surgical robotic procedures including an end effector base, an instrument adaptor, and an instrument;

FIGS. 21A-21C show the active end effector base, the internal components thereof, and a hall sensor configuration for determining the location of the instrument adaptor relative to the end effector base, respectively, according to one embodiment;

FIGS. 22A-22B show further details of the instrument adaptor relative to the end effector base according to one embodiment;

FIGS. 23A-23B show an instrument adaptor with a drive assembly for rotating the instrument and a wireless communications system, respectively;

FIG. 24 shows an oscillating drill adaptor according to one embodiment;

FIGS. 25A-25B show perspective and section views, respectively, of the distal end of a scrill adaptor according to one embodiment;

FIG. 26 shows a sterile drape assembly with a plastic cap encapsulating an electrical connector and a sterile drape attached to the plastic cap according to one embodiment;

FIGS. 27A-27B show the power and data transmission components for the palm assembly and end effector, respectively;

FIGS. 28A-28B show perspective and side views, respectively, of the electrical connector including a printed circuit board with pogo pins and a magnet for connecting the end effector to the robot arm according to one embodiment;

FIGS. 29A-29B show exploded and assembled views, respectively, for attaching the sterile drape assembly to the mounting flange on the robot arm and the end effector according to one embodiment;

FIGS. 30A-30B show disassembled and assembled views, respectively, of an alternative sterile drape assembly with an intermediate clamp according to one embodiment;

FIGS. 31A-31B show front and back views, respectively, of the power and data transmission connectors on the sterile drape assembly according to one embodiment;

FIGS. 32A-32C show examples of an O-ring seal and a metal seal, respectively, for sealing the intermediate clamp to the drape;

FIG. 33 shows automatic ultrasound registration according to further embodiments;

FIG. 34 shows ultrasound-enhanced tracking systems according to further embodiments;

FIG. 35 shows low profile ultrasound sensors attached to the patient's spine, which have QR codes to serve as machine vision identifiers and tracking fiducials for each sensor according to one embodiment;

FIG. 36 shows ultrasound guided tracking according to further embodiments; and

FIG. 37 shows the surgical robotic system of FIG. 1 having two surgical arms where one or both arms include ultrasound transducers according to one embodiment.

### DETAILED DESCRIPTION OF THE INVENTION

Embodiments of the disclosure are generally directed to surgical robotic systems and related devices and methods. In particular, the surgical robotic systems may include integrated real-time surgical navigation with multiple surgical arms configured to assist a user with one or more surgical tasks. End effectors may be attached to each surgical arm to guide the trajectory of specialized surgical instruments and perform the desired surgery. For example, the robotic system may include a pair of surgical arms, which guide the instruments to follow the trajectories specified by the user. A multi-arm system may provide opportunities to greatly expand the capabilities of computer-assisted technology in surgery. The multiple surgical arms allow the robotic system to assist with more surgical procedures and improve the accuracy of the procedures. The advanced multi-arm highly automated platform may allow for simultaneous interaction by one or more surgeons, technicians, and the patient.

The surgical robotic systems may be configured for full navigation and accurate alignment during spine surgery. The surgical robotic systems may allow for locating anatomical structures in open or minimally invasive surgical (MIS) procedures and navigation of surgical instruments and devices in real-time. For example, the surgical arms and attached end effectors may be used during spinal surgery to position and install pedicle screws, interbody implants, or perform or other surgical techniques. Although generally described herein with reference to performing spinal surgery, it will be appreciated that the systems and methods described herein may be applied to other orthopedic locations in the body as well as other medical procedures, such as trauma applications, cranial procedures, and oncology applications.

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures may have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

### Multi-Arm Surgical Robotic System

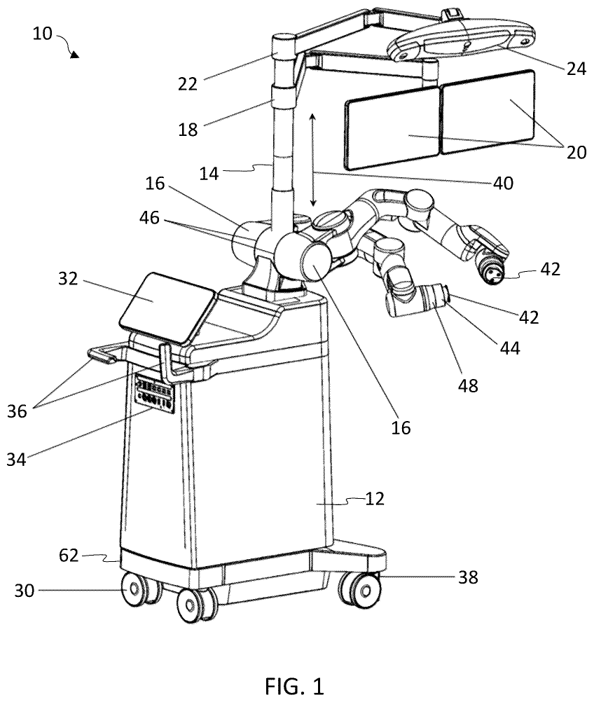

Turning now to the drawing, FIG. 1 illustrates a multi-arm surgical robotic system or platform 10 in accordance with one embodiment. The multi-arm surgical robotic system 10 is configured to complete multiple surgical tasks, simultaneously or sequentially, which may improve the accuracy of the overall procedure and reduce surgical time. Surgical robotic system 10 may include, for example, a robotic base station 12, an arm positioner 14 attached to the base station 12, and multiple arms 16, 18, 22 attached to the positioner 14. Two or more surgical arms 16 may help to guide instruments or perform the surgical tasks, for example, using an end effector. A monitor arm 18 is configured for supporting one or more displays or monitors 20. A camera arm 22 is configured for supporting one or more cameras 24, for example, navigation cameras for detecting and tracking markers, such as active and passive markers. Unlike other robotic systems which may utilize a separate viewing/control station or separate camera stand/station, all of the system components are integrated into a single mobile unit for robotic system 10. The integration of all components into one mobile platform may improve usability and accuracy of the system 10, while also reducing the overall footprint in the operating room.

The robotic base station 12 may include a mobile cabinet or portable frame, for example, on casters or wheels 30. The base station 12 houses an on-board computer or computing unit for controlling all functionality of the robotic system 10. The on-board computer may include a central processing unit (CPU), memory, and an input/output interface. The central processing unit carries out the instructions of a computer program or software by performing arithmetical, logical, control, and input/output (I/O) operations specified by the instructions. The memory may include volatile and non-volatile memory storage that temporarily or permanently store data and instructions that are currently in use or will be needed by the central processing unit. This may include, for example, random access memory (RAM), read-only memory (ROM), and storage devices like hard drives. The input/output interface allows the computer system to interact with the user, take in information, and deliver results, and may include devices such as a monitor, keyboard, mouse, network interface for internet connectivity, and so forth.

As shown in the embodiment of FIG. 1, the multi-arm surgical robotic system 10 may include one or more user interfaces, such as displays or monitors 20, 32 including touchscreen displays, which may be operated by one or more surgeons or other users. Before or during the medical procedure, two-dimensional (2D) and/or three-dimensional (3D) images, such as computed tomography (CT) scans, may be taken of a desired surgical area of a patient and provided to the on-board computer. The surgeon may use the images to program a desired point of insertion and trajectory for one or more surgical instruments to reach a desired anatomical target within or upon the body of the patient. The desired point of insertion and trajectory may be planned on the images, which may be displayed on monitor(s) 20, 32. The system 10 includes 2D & 3D imaging software that allows for preoperative planning, navigation, and guidance throughout the surgical procedure. Further details of surgical robotic and navigation systems can be found, for example, in U.S. Patent Publication No. 2019/0021795 and U.S. Patent Publication No. 2017/0239007, which are incorporated herein by reference in their entireties for all purposes.

In one embodiment, a pair of monitors 20 may be affixed to monitor arm 18, which is part of the sterile field. The pertinent information may be displayed and manipulated by the surgeon(s) on touchscreen monitors 20 before or during the procedure. Unlike systems with only a single monitor viewable by the surgeon, the dual monitor display may provide access to a secondary surgeon or assistant, and may allow for separate control of each of the respective surgical arms 16. The monitors 20 may be arranged side-by-side, back-to-back, or in another suitable configuration for accessibility and visibility by the user(s). The base station 12 may further include a cabinet-mounted terminal or touchscreen control display 32. The cabinet-mounted touchscreen display 32 may be accessible to a user when the system 10 is docked, during transport, or during the procedure. During the surgery, cabinet display 32 may be used for non-sterile user control or observation, for example, by an assistant. It will be appreciated that one or more of the displays 30, 32 may be supplemented or replaced with an optional wireless tablet or other suitable device.

The surgical robot system 10 may also utilize a camera 24, for example, affixed to camera arm 22. The camera arm 22 is configured to move, orient, and support the camera 24 in a desired position. The camera 24 may include any suitable camera or cameras, such as one or more infrared cameras (e.g., bifocal or stereophotogrammetric cameras), able to identify, for example, active and passive tracking markers in a given measurement volume viewable from the perspective of the camera 24. The tracking markers may be arranged in a specific array or pattern, which may help to identify the instrument, for example. In an exemplary embodiment, the camera 24 is a machine vision navigation camera configured to capture visual data from the tracking markers, which may be present on the system 10, on the instrument(s), affixed to the patient, or in any other suitable locations for tracking and navigating the surgical procedure. The camera 24 may scan the given measurement volume and detect the light that comes from the markers in order to identify and determine the position of the markers in three dimensions. For example, active markers may include infrared-emitting markers that are activated by an electrical signal (e.g., infrared light emitting diodes (LEDs)), and passive markers may include fiducials, retro-reflective markers (e.g., spheres or discs) that reflect infrared light (e.g., they reflect incoming IR radiation into the direction of the incoming light), for example, emitted by illuminators on the camera 24 or other suitable device. In one embodiment, the tracking markers may include machined fiducials, reflective discs, reflective spheres, and/or active LEDs, which are visible directly or through surgical draping. The location, orientation, and position of structures having these types of markers may be provided to the on-board computer, which may be shown to the user on the display(s) 20, 32. The navigation camera 24 tracks the positions in real time and provides image(s) on the monitors 20, 32, along with the patient's images, for example, to provide guidance to the surgeon during the operation.

The base station 12 may also include a connector panel 34, which includes external connection ports for various devices, such as an equipotential terminal, a foot pedal connector, a camera connector port, an HDMI connector, an ethernet connector, dual USB 3.0 ports, and the like. It will be appreciated that any suitable hardware, software, or combinations thereof may be implemented for carrying out the operation and functionality of the robotic system 10.

The robotic base station 12 may include a motorized propulsion and positioning system to transport the robotic system 10. In this manner, one or more base wheels 30 may be powered and steerable by the user before or during the procedure. The motorized propulsion and positioning system may include two primary modalities. In a first configuration, the user is able to transport and position the robotic system 10 via one or more handles 36. Powered base motion may be controlled by force input from the user on handles 36. Force feedback may be measured at the handles 36, for example, to regulate the direction and speed of movement. In a second configuration, the user may utilize smart positioning in the operating room for position recall and reachability adjustments. Smart positioning may be achieved via encoders at the wheels 30 and relative positioning tracking with navigation camera 24. Relative tracking with camera 24 may be achieved, for example, with patient reference tracking, simultaneous localization and mapping (SLAM), and/or machine vision. The smart positioning may allow for intraoperative positioning of the system 10, for example, to account for long implant constructs or complex cases. The base station 12 may include a braking and/or stabilizer system 38, to secure the base 12. The stabilizer system 38 may be rigidly fixated to stabilize the system 10 in the operating room and lock the base 12 to the ground during surgery. Stabilization may be engaged and retracted via motor power and manually by the user. The braking system may also be used when the system 10 is powered off to assist with transport.

The arm positioner 14 is affixed to the base station 12 and controllable via the on-board computer. In one embodiment, the arm positioner 14 may include a vertical column, which provides for telescoping movement along z-axis 40, thereby functioning as a prismatic joint. Thus, the arm positioner 14 may extend or contract in a vertical direction, thereby moving one or more arms 16, 18, 22 of the system 10. As shown in this embodiment, the surgical arms 16 may be coupled to the arm positioner 14 near the base station 12 and the monitor and camera arms 18, 22 may be located toward a distal end of the arm positioner 14. In this manner, vertical movement of the arm positioner 14 may provide for movement of the monitor(s) 20 and camera(s) 24 along z-axis 40. It will be appreciated that other suitable configurations may be used to position the respective robot arms 16, 18, 22.

One or more surgical arms 16 may be provided to perform a wide range of motions and adjustments, for example, mimicking the movements of the human arm, hand, and/or fingers and closely replicating the dexterity and precision of a skilled surgeon. In one embodiment, the surgical arms 16 include a pair of left and right surgical arms arranged about the bottom of the arm positioner 14. Each surgical arm 16 may include a plurality of arm segments or links interconnected by various types of joints. Each joint may allow for specific types of movement or offer specialized motion. The joints may include rotary joints, prismatic joints, spherical joints, universal joints, cylindrical joints, planar joints, or other suitable joints that contribute to the arm's range of motion, flexibility, and reach. In the embodiment shown, the system 10 includes left and right surgical arms 16, which each allow for movement with seven degrees of freedom (7 DoF). For example, the movement may include three translational movements (along the x, y, and z axes), three rotational movements (around the x, y, and z axes), and an additional rotation or translation for imparting high precision and dexterity. It will be appreciated that the surgical arms 16 may be configured to have any suitable orientation or movement allowing each arm 16 to move forward/backward, left/right, up/down, yaw left/right, pitch tilt up/down, roll around its own axis, or otherwise translate or rotate for complex movement. The surgical arms 16 may be configured with zero backlash to ensure the movements are highly precise, accurate, and directly reflective of the surgeon's commands without any delay.

The distal end of each surgical arm 16 includes an end effector interface 42 for securing the end effector to the end of the surgical arm 16. The end effector is a device or tool, which attaches to the end of the robotic surgical arm 16 to interact with the surgical site. In some cases, the end effector may include a guide tube to provide precise positioning of instruments placed therethrough. In other cases, the end effector may include an active or workable instrument, such as a retractor for retracting soft tissues, which is controlled by the system 10 or manually. The end effector may be provided as a separate component, which is sterilized prior to use. The end effector interface 42 may include mechanical and/or electronic coupling of the end effector to the distal end of the surgical arm 16. The end effector interface 42 includes a power and communication interface for the end effector. The end effector interface 42 allows for a rigid connection of the end effector to the surgical arm 16 through the sterile drape.

The end effector may be configured to guide or hold an integrated or separate navigated instrument. For example, the end effector may include a tubular element or guide tube aligned along a planned trajectory. A separate navigated instrument may be positioned through the guide tube and along the planned trajectory to perform a given function. For example, the navigated instruments may include drills, taps, drivers or other instruments for inserting screws, for example. The navigated instruments may further include dilators, disc preparation instruments (e.g., curettes, Cobb elevators, osteotomes, rasps, scrapers, etc.), trials, retractors/distractors, inserters, and other instruments for installing interbody implants, for example. It will be appreciated that any suitable instruments may be used for the designated surgical procedure.

Each surgical arm 16 may include one or more load cells 44, 46 configured to monitor and measure forces applied to the surgical arm 16. A distal load cell 44 may be provided near the free end of each surgical arm 16. For example, a 6-axis load cell 44 may be located at the end effector interface 42, which provides collaborative, ad hoc move mode when the user moves the arm 16 by directly applying force to the end of the arm 16 or end effector. A base load cell 46, such as a 6-axis load cell 46, may also be provided in each arm 16 near the connection to the arm positioner 14 to provide real-time feedback to the control system of the robot 10.

Each surgical arm 16 may include a ring of information (ROI) 48 for status indications. Each ring of information 48 may provide independent information about the status of each respective arm 16. For example, the ring of information 48 may provide individual colors, such as green for system ready, red for error, yellow for user action, etc., which conveys information to the user. The information ring 48 may also blink or provide other visual indicators to the user(s). The ring of information 48 may be located anywhere along each arm 16 or in another suitable location.

The monitor arm 18 is attached to the positioner 14, for example, near the top of the positioner 14. The monitor arm 18 includes a motorized arm with a plurality of arm segments interconnected by various types of joints. The motorized monitor arm 18 may be controlled by the system 10 and/or the user for optimal visibility of the monitor(s) 20. In one embodiment, the monitor arm 18 is connected to the positioner 14 at a rotary joint, the arm segments are interconnected by a duplex hinge joint, and the monitors 20 are coupled to the free end of the monitor arm 18 via two rotary joints, respectively. As shown, the monitor arm 18 may allow for movement with four degrees of freedom (4 DoF). For example, the movement may include x, y, z, and yaw with the folding butterfly providing angle control. It will be appreciated that the monitor arm 18 may be configured to have any suitable orientation or movement allowing the arm 18 to support and position in the monitors 20 for optimal visibility.

The camera arm 22 is attached to the positioner 14, for example, at the distal-most end of the positioner 14. The camera arm 22 includes a motorized arm with a plurality of arm segments interconnected by various joints. The motorized camera arm 22 may be controlled by the system 10 and/or the user for optimal line of sight of the camera 24 throughout the procedure. In one embodiment, the camera arm 22 is connected to the positioner 14 at a rotary joint, the arm segments are interconnected by a duplex hinge joint, and the camera 24 is connected to the free end of the arm segments with a pivot or tilting joint. As shown, the camera arm 22 may allow for movement with six degree of freedom (6 DoF). For example, the movement may include x, y, z, yaw, pitch, and tilt. In one embodiment, the navigation camera 24 is mounted to arm 22 in a SCARA configuration (Selective Compliance Articulating Robot Arm) with a prismatic vertical joint for height adjustment followed by two in-plane revolute joints for x-y positioning. The camera itself may have 3-axis of orientation control (pan, tilt, roll) totaling 6-axis camera positioning. All joints may be motorized and use absolute, single-turn encoder feedback, allowing the system 10 to know camera position immediately on system power-up without the need for a homing routine. The camera arm 22 may be bimodal, active and passive, meaning it can be positioned either robotically or by manual surgeon interaction, achieving the collaborative approach for the system 10. Unlike systems which provide the camera on a separate stand, system 10 incorporates the camera 24 into a single cart solution. This may help to improve line of sight for the camera 24, which can only navigate if the camera 24 is able to see the patient reference and instrument of interest. The motorized camera arm 22 also allows for adjustment of the camera 24 during the procedure, which minimizes possible line of sight disruptions and removes the need for any manual positioning of the camera.

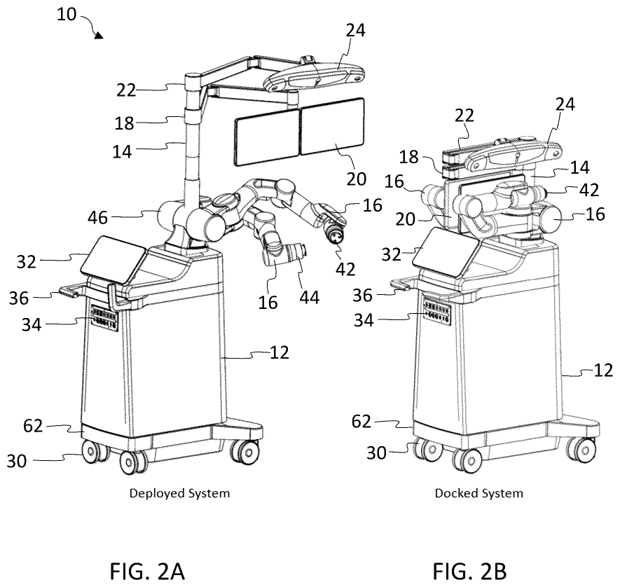

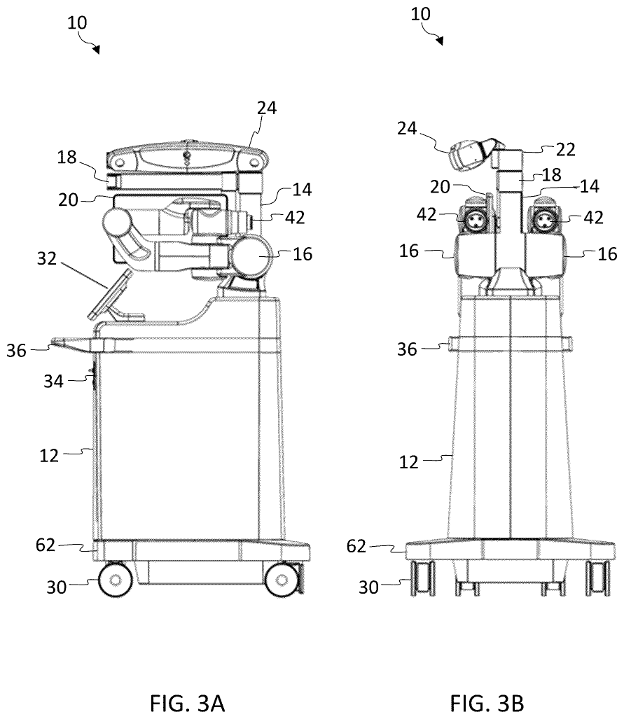

Turning now to FIGS. 2A-2B and 3A-3B, the multi-arm surgical robotic system 10 may have a deployed position and a docked position. In the deployed position shown in FIG. 2A, one or more of the robot's arms 16, 18, 22 are extended and positioned for active participation in a surgical procedure. In the deployed configuration, the surgical arms 16 may be arranged to provide optimal access to the surgical site, the camera arm 22 may be extended to provide optimal line of sight for the machine vision navigation camera 24, and the monitor arm 18 may be extended for optimal viewing and participation with the touchscreen monitors 20. In the docked position shown in FIG. 2B, all of the arms 16, 18, 22 are folded in such that the surgical robot 10 is in a compact configuration, for example, for transport or storage. The cabinet touchscreen display 32 remains accessible while the system 10 is docked. With further emphasis on the docked system 10 shown in FIGS. 3A-3B, all robotic arms 16, 18, 22 dock in compact form for easy transport and to enable selective deployment of individual arms 16, 18, 22. This arrangement allows for all four arms 16, 18, 22 in the system 10 to dock in a compact form factor, and allows the user to selectively deploy subsets of the arms 16, 18, 22 depending on the specific use case. For example, a use case where one surgical arm 16 is needed, and the video output is sent to large operating room (OR) monitors rather than using the integrated monitors 20 may result in the monitors 20 staying retracted while one surgical arm 16 is deployed. Deployment and docking may be motorized and automated, which enables a simple and elegant setup where it might otherwise be overwhelming or complicated.

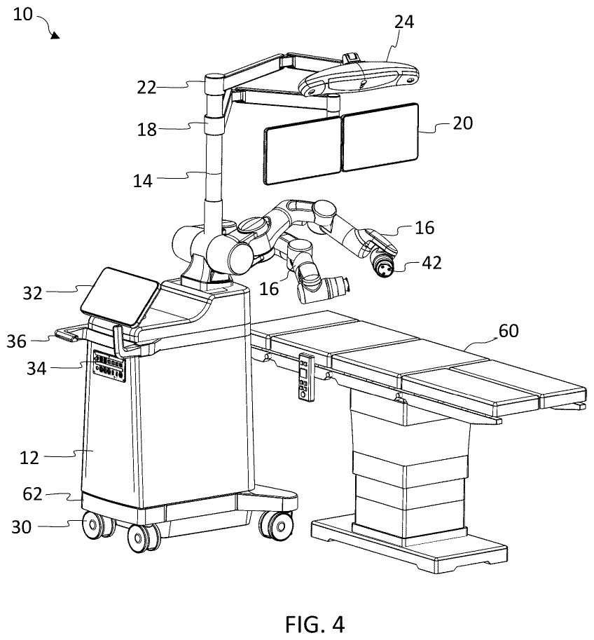

Turning now to FIG. 4, system positioning at the operating room table 60 is flexible and may be based on surgeon preference for a given procedure. The multi-arm surgical robotic system 10 may be positioned next to, or across from the surgeon, and may also be located toward the foot or head of the patient. In all these combinations, the surgical arms 16 have a large working volume on both sides of the table 60 without moving the system base 12, and the monitors 20 and camera 24 may be positioned along the midline of the table 60. This camera position limits line of sight issues, and the monitor position is more ergonomic for the surgeon than other systems. In the embodiment shown in FIG. 4, both surgical arms 16 are the same in length and configuration. Alternatively, the surgical arms 16 may be different. The length of one arm 16 may be increased, for example, through an attachment at the end between the end of the arm 16 and the end effector, or two distinct arm configurations may be provided. This differentiation in length and/or type may lead to a primary arm and a secondary arm when positioning in the OR and deploying for procedures.

### Base and Drive Wheels

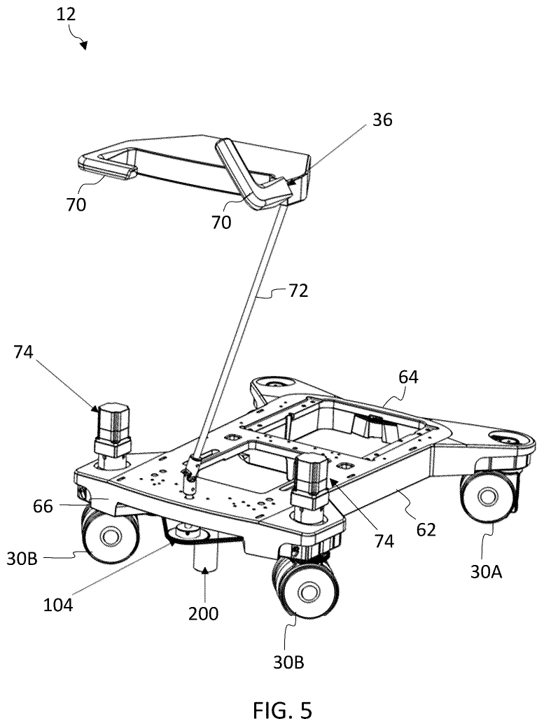

Turning now to FIGS. 5-19, the mobile robotic base station 12 is described in more detail. One or more wheels 30 may be equipped with motors 74 to assist with propulsion, braking, and directional control. Unlike systems which are unpowered and/or without directional control, system 10 includes a powered base station 12. For unpowered systems, the mass of the system (e.g., 800 lb system weight) as it relates to motion initiation, cessation, and directional changes are fully controlled and powered by the user. On level ground, this is a minimal hardship, but for transportation on carpet, up and down ramps, tight maneuvers and rapid start/stops can make manual control challenging at times. Accordingly, system 10 may include automated motion and directional control of the robot base 12 to address these shortcoming.

As shown in FIG. 5, the robotic base station 12 may include a dolly or bottom tray 62. The bottom tray 62 may be configured to support the frame, cabinet, or enclosure of the base 12, which houses the on-board computer and any other components, and supports the robotic arms 16, 18, 22. The bottom tray 62 may have a front end 64 and a rear end 66. The front end 64 may generally designate the front of the robot system 10 and may enter a space first when the robot 10 is being transported or moved. The rear end 66 may generally designate the back or user side of the robot system 10. A plurality of wheels 30 may be attached to the underside of the bottom tray 62 to allow for movement of the robot 10. For example, four wheels 30, two front wheels 30A and two rear wheels 30B, may be attached at the four corners of the bottom tray 12 for enhanced stability and maneuverability. The wheels 30 may include casters, omni-wheels, track wheels, spherical casters, or other devices for locomotion.

The bottom tray 62 is controlled by the user input handle 36 which may include a pair of grips 70 for ergonomic handling. The handle 36 is connected to the bottom tray 62 by steering shaft 72, enabling precise and coordinated steering actions by the user. Direction and movement of the bottom tray 62 may be directly influenced by the user's input through the handle 36, providing a seamless and intuitive control mechanism for navigating the robot system 10.

One or more wheels 30 may incorporate motors 74 to aid in movement, stopping, and steering. In one embodiment, motorized propulsion may be integrated into the left and right rear caster wheels 30B. In other words, the two rear wheels 30B may be steerable casters with motorized propulsion, while the front wheels 30A are unpowered. The two rear wheels 30B may also include duplex wheels with a combination of driven and non-driven wheels. The motors 74 may include stepper motors, DC brushed motors, brushless DC motors, servo motors, induction motors, gear motors, or the like. In one embodiment, motor 74 includes a stepper motor with reduction gearing, which is paired with the caster housing. The powertrain may be sized to provide adequate torque for starts on an incline, as well as sufficient speed for comfortable travel on level ground.

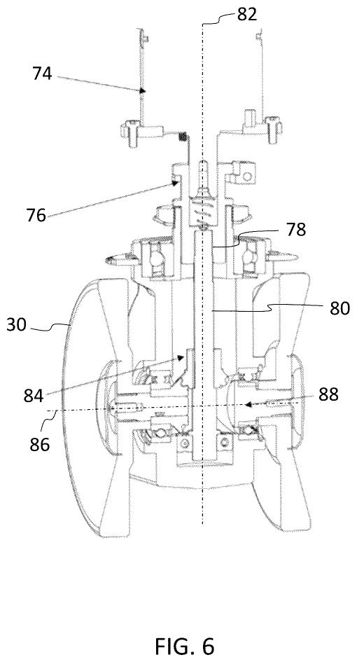

As best seen in FIG. 6, a cross-section of a motorized wheel assembly is shown according to one embodiment. The stepper motor 74 with reduction drive is mounted to the top of the bottom tray 62. The stepper motor 74 may move in discrete steps offering precise control over position and speed without the need for feedback systems. The motor 74 is configured to turn wheel 30 and provide precision control over rotation of the wheel 30. Force sensing, integrated into the handle 36, may be an input parameter for the motor drive. An output reduction gearbox may be attached to the motor 74, which reduces the speed and increases the torque output of the wheel 30. Each of the powered wheels 30B may be connected to the power unit output drive via a splined shaft 76 with intermediate coupling. The splined shaft 76 may include a recess 78 with splines configured to engage a central shaft 80. The spline shaft 76 and central shaft 80 may be aligned along vertical axis 82. The splined coupling connects the output shaft of the reduction gearbox to the steering mechanism of the wheel itself. The central shaft 80 is configured to engage gearing 84 to transmit torque to the wheel 30. The output drive miter gearing 84 may include a set of bevel gears arranged to transmit power from the gearbox to the wheel 30 at a right angle. The wheel 30 may be supported on an axle to permit rotation about wheel axis 86. To avoid the need for differential action at the drive output, a single wheel 30 may be being driven by the motor 74. The duplex wheel 88 is allowed to spin freely. The freewheeling non-drive wheel 88 on bearings may not be directly driven by the motor 74 to allow for easier turning and maneuverability as non-drive wheels may rotate independently of the motorized motion. At a system level, the power system may be configured so that the driven wheels 30B are on the outside of the cart 12. This enables the cancelation of moment loads about the steering axis of each wheel 30. The wheel assembly may provide for precise control over the wheel's motion, and the stepper motor 74 may provide for controlled incremental movements and exact positioning of the robotic system 10.

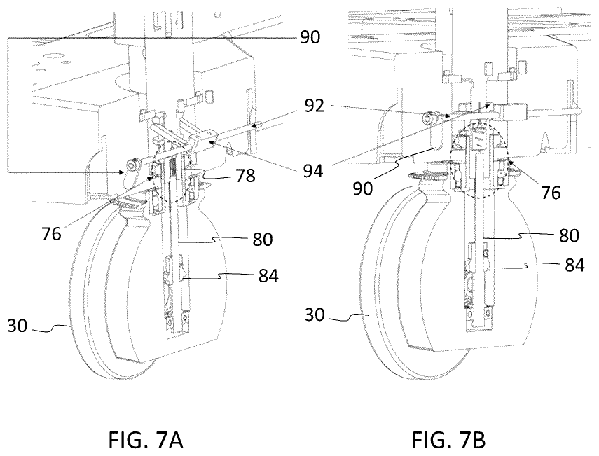

With further emphasis on FIGS. 7A-7B, a manual override assembly is shown according to one embodiment. The manual override assembly option may be provided to decouple the motor output from the driven wheel. This allows the system to be moved manually without back driving the motor through the gearbox. This is a safety feature useful for situations when the system must be moved in the absence of power, as well as risk mitigation for an unanticipated power failure in a patient setting. In one embodiment, the manual override may be actuated via a release lever 90 having a release shaft 92 and a release fork 94. The splined shaft 76 may be spring loaded such that the splined shaft 76 presses downward and the end of the central shaft 80 fits into the splined recess 78. When the release shaft 92 is twisted, the release fork 94 disengages the splined shaft 76 from the central shaft 80 by lifting the splined shaft 76 upward. In this manner, the end of the central shaft 80 disengages from the splined recess 78. FIG. 7A shows the spline disengaged, thereby decoupling the splined shaft 76 from the central shaft 80 and preventing any powered movement. FIG. 7B shows the spline engaged, thereby engaging splined shaft 76 with the central shaft 80 and permitting powered movement. The release configuration may be adapted to integrate into the wheel assembly to meet user needs and product aesthetics.

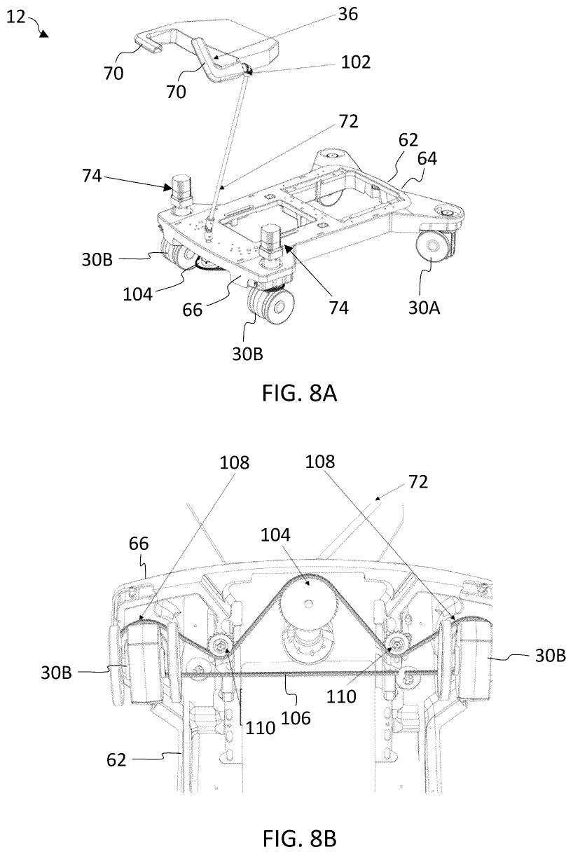

Turning now to FIGS. 8A-8B, directional control of the system 10 is shown according to one embodiment. The drive direction of the caster(s) 30 may be controlled via input from the user at the handle 36. Steering may be accomplished with a bevel gear pair 102 in the handle 36 coupled to the steering shaft or input shaft 72 for the steering system. The bevel gears 102 may include cone-shaped gears configured to transmit rotational motion between intersecting axes, for example, at a 90-degree angle. When the user turns the handle 36, the bevel gear 102 within it turns and engages with the bevel gear on the steering shaft 72. The bevel gears 102 provide for motion conversion as it allows maximum flexibility in shaft angle and position. The input shaft 72 may be coupled to a steering sprocket 104 via a universal joint to align with the vertical shaft.

Rotation movements between casters 30 may be synchronized via chain drive 106. The steering sprocket 104 may be coupled via chain 106 to sprockets 108 fitted to each of the drive wheels 30. The sprockets 104, 108 may include wheels with teeth or cogs configured to mesh with chain 106 to transfer motion. Combined with tensioners and idlers 110, as needed, the steering system provides synchronized steering control to both rear casters 30B via user input at the handle 34. The steering system may be configured to drive a 90° rotation of the casters 30 to enable pure lateral movement of the system. This functionality may be useful for fine positioning of the robot 10 adjacent to the patient prior to stabilization deployment as well as for adjustments mid-procedure. One or more position encoders may be located at the input sprocket 104, wheel sprockets 108, or another suitable location to track the respective position, speed, or direction and provide feedback to the system 10.

Force sensing, integrated into the handle 36 to drive the motors 74 forward can differentiate unequal loading across the handle 36 and use this sensing to infer desired direction of travel. The left and right wheel motors 74 may be asymmetrically driven to assist in direction changes. Additionally, an absolute position encoder may be integrated into the steering linkage such that steering angle may be an input parameter for motor drive commands. Steering angle as an input parameter, combined with handle force sensing allow for the incorporation of transport features optimization for the user. Transport features may include, for example, automatic reduction of speed when turning a corner and asymmetric motor drive commands to assist in tight-space maneuvering. By physically steering the wheels while also influencing direction of travel, the system 10 provides improved ease of use and enhanced control.

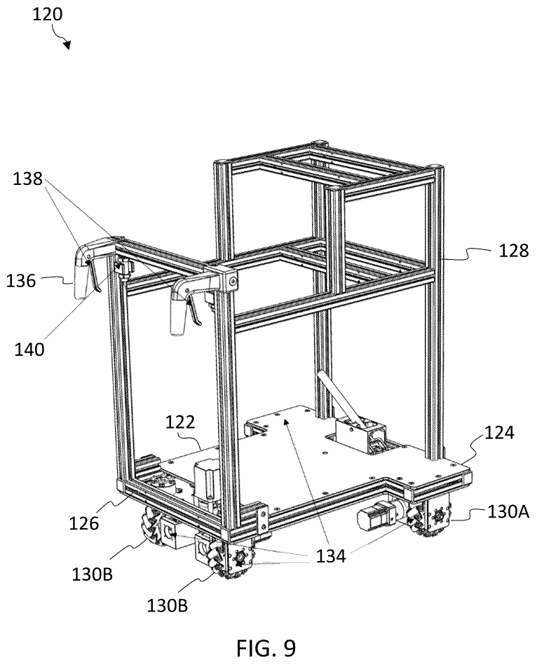

Turning now to FIG. 9, an alternative moveable base station 120, which merges propulsion and steering functions into the same set of hardware, is shown according to one embodiment. In this embodiment, the robotic base station 120 may include a dolly or bottom tray 122 configured to support a frame 128, which houses the on-board computer and any other components, and supports the robotic arms 16, 18, 22. The bottom tray 122 may have a front end 124 and a rear end 126. The front end 124 may generally designate the front of the robot system 10 and may enter a space first when the robot 10 is being transported or moved. The rear end 126 may generally designate the back or user side of the robot system 10. A plurality of wheels 130 may be attached to the underside of the bottom tray 122 to allow for movement of the robot 10. For example, four wheels 130, two front wheels 130A and two rear wheels 130B, may be attached at the four corners of the bottom tray 122 for enhanced stability and maneuverability. In this embodiment, the wheels 130 may include omnidirectional wheels, omni-wheels, or mecanum wheels, which are configured to move in any direction. Propulsion and steering may be accomplished with an arrangement of four drive units 134, for example, each housing a motor 148 with reduction gearbox, right angle drive 146, and mecanum wheel 130.

Propulsion and steering commands may be input via a human-machine interface located in the system handle 136. The handles 136 may be attached to an upper portion of the frame 128. The handle interface 136 may include a two element safety system 138 for movement, which is normally off, and an array of multi-directional force sensors 140 structurally integrated in the handle 136. User force, applied to the handle 136 and measured by the sensors 140 is converted into desired travel velocity (speed and direction). Movement commands are processed by the on-board computer and output to the four mecanum drive units 134 to enable holonomic motion of the system. Drive control system architecture may be adapted based on the functional requirements of the system. For example, drive control may be executed as a basic open-loop stepping system, a sophisticated closed-loop servo system, or an iteration in between. In one embodiment, the individually controlled wheels 130 allow the system to move laterally along the patient bed without having to manipulate steering levers or forcefully ensure the correct direction of travel. The wheels 130 allow the system to rotate a full 90° and change travel mode from straight-line to lateral while maintaining powered motion. This may be useful when positioning for X-ray equipment or multi-level cases that require intermediate base repositioning for reachability. Additional software control may include a lateral mode for such instances that locks out forward/reverse movements when enabled and only permits lateral translation of the cart.

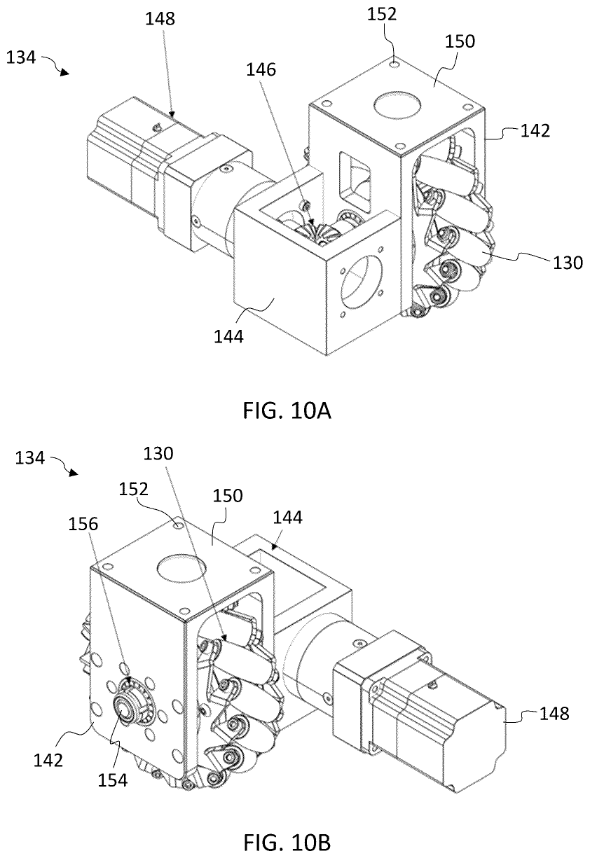

FIGS. 10A-10B show mecanum drive units 134 configured for structural rigidity and configuration flexibility in more detail. Each drive unit or module 134 may include an outer housing 142 for holding the wheel 130, an offset housing 144 for supporting a right angle drive 146, and a motor 148 with reduction gear box for powering the wheels 130. The drive module 134 is configured to keep the mecanum wheel 130 orthogonal to the drive surface and primary axis of rotation in order to ensure smooth, repeatable, and predictable control. The wheel housing 142 may include a broad, flat mounting surface 150 with shoulder screw interface 152 to control these critical orientations. For example, shoulder screw openings 152 may be located at each corner of the mounting surface 150 to attach the unit 134 to the underside of the bottom tray 122. The housings 142, 144 may be reversible to allow the drive unit 134 to be configured for left, right, front, and back configurations without the need for location-specific parts beyond the properly handed mecanum wheel 130. The wheel 130 may be mounted in housing 142 with axle 154. The axle 154 may include a robust steel axle suspended between two oversized tapered roller bearings 156 with sufficient preload imparted via locknut to eliminate axial play along the wheel axis. The offset housing 144 supports the right angle drive 146 and the motor 148. The motor's axis of rotation is perpendicular to the axis of the wheel 130. The right angle drive 146 may include bevel gears, which can transmit power from the motor 148 to the wheel 130. Four mecanum drive units 134 may be provided at the four corners of the bottom tray 122, thereby enabling full propulsion and steering of the system.

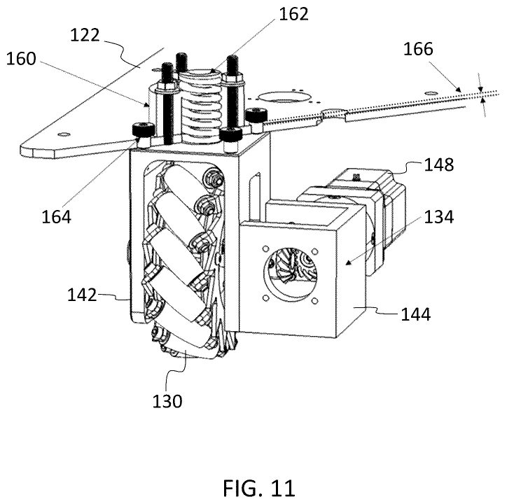

FIG. 11 depicts a suspension system for the drive units 134 according to one embodiment. All mecanum wheels 130 may participate together in any system movement. Thus, continued contact of all wheels 130 with the ground allows for optimal performance. Planar ground contact with four points is statically indeterminate, meaning that the system may be stable and stationary on the ground with one wheel lifted, for example, which may be encountered by traversing an uneven floor. Accordingly, a suspension system may be employed to ensure that all wheels 130 remain in constant contact with the ground. For one or more of the wheels 130, the wheel(s) 130 may utilize a suspension system having a spring housing 160 for holding a compression spring 162. The spring housing 160 may be secured to the base tray 122 with shoulder screws 164 receivable in corresponding openings 152 in the drive unit 134. In one embodiment, the two front wheels 130A are fixed to the base 122, and the two rear wheels 130B are preloaded against the ground with the coil springs 162. The preload may be dialed-in, for example, based on system weight and distribution. For small dips and unevenness in the floor, where a fixed wheel setup would normally encounter a wheel lifting off, the suspended wheels 130 may have a given travel 166 (e.g., 3.5 mm of travel). The amount of travel 166 permits the wheel 130 to move downward to meet the low spot while still being preloaded against the ground with sufficient spring force to generate propulsion loads at the wheel-ground interface. The entire drive module 134 may articulate this distance, in pure vertical motion piloted by the shoulder screw interface 152, 164 with the base 122. The suspension functionality may be incorporated into one or more of the wheel units 134 to ensure adequate ground contact of the wheels 130.

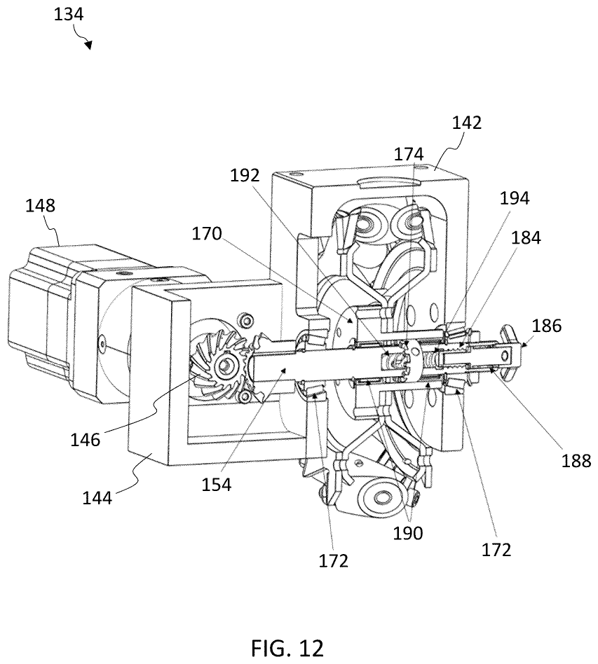

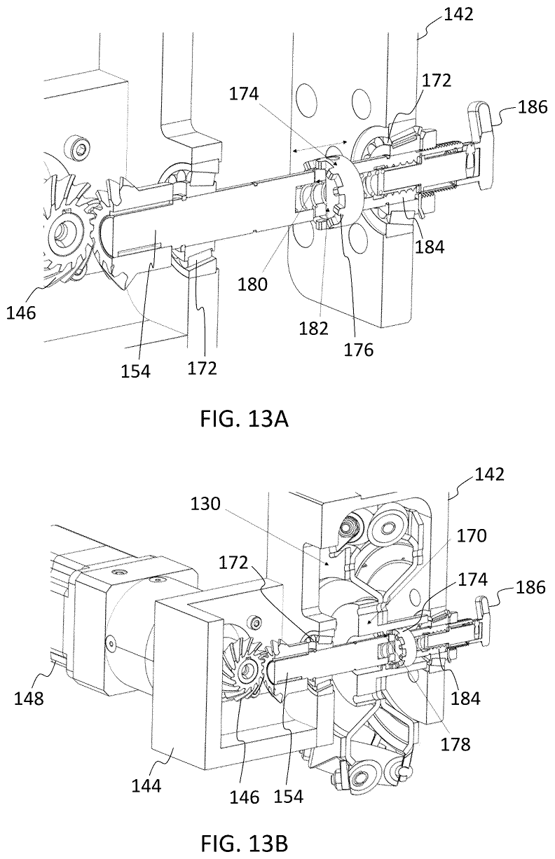

Turning now to FIGS. 12-15, a system for decoupling the wheel 130 from the drive unit 134 is shown according to one embodiment. The decoupling system allows for a quick, safe mechanism for converting to a non-powered push-style cart to abate any electrical or drive system failure risks as they relate to system mobility and bail-out. The decoupling version of the drive module 134 is the same as the non-decoupling version except the drive axle assembly is modified to accommodate the decoupling system to decouple the wheel 130 from the drive axle 154. In this embodiment, the hub 170 onto which the wheel 130 mounts is separate from the motor-driven axle 154. The decoupling hub 170 includes a central bore for receiving the end of the drive axle 154. The mecanum wheel 130 is mounted to the decoupling hub 170. The wheel 130 is allowed to rotate about the drive axle 154 on the primary bearings 172. The decoupling hub 170 is configured to engage and disengage a decoupling key 174. As best seen in FIGS. 13A-13B, the decoupling key 174 may include cylindrical part with a plurality of teeth 176 configured to engage with the corresponding teeth 178 on the decoupling hub 170. The toothed decoupling key 174 may be pinned to the slot 180 in the drive axle 154 via pin 182, thereby constraining it rotationally, but allowing for translation within the extent of the slot 180. The decoupling key 174 is attached to an actuator 184, which is internally threaded.

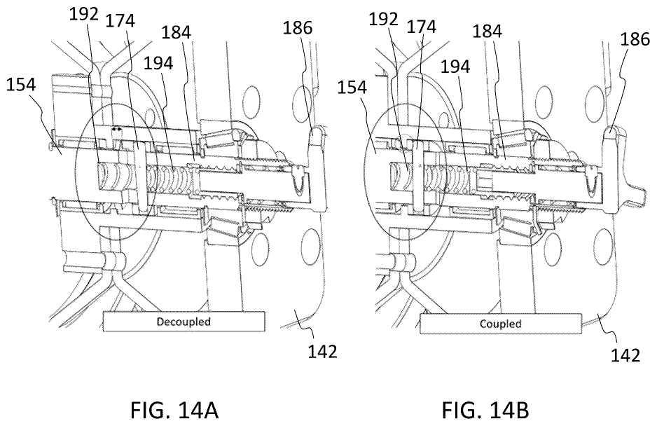

The wheel 130 may be coupled and decoupled from the drive motor 148 via a selector knob 186. The selector knob 186 may be spring-loaded with a return spring 188. When turned by the selector knob 186, the actuator 184 moves in and out, engaging or disengaging the decoupling key 174 from mating features in the decoupling hub 170. As shown in FIG. 14B, when the decoupling key 174 is engaged, it couples the mecanum wheel 130 to the drive axle 154. As shown in FIG. 14A, when the decoupling key 174 is disengaged, the mecanum wheel 130 is allowed to rotate freely about the drive axle 154 on the freewheel bearings 190. The gap between actuator 184 and decoupling key 174 may be bounded by a pair of balancing springs 192, 194 that load against the pin 182 between the decoupling key 174 and axle shaft 154. The first spring 192 may include a low force spring, which helps to push the decoupling key 174 out of the decoupling hub 170. The second spring 194 may include a high force spring, which acts as a buffer between the actuator 184 and decoupling key 174. If the key-hub teeth 176, 178 are not aligned, the selector knob 186 can still be operated normally by the user, but the actuation can take place when teeth 176, 178 in the key 174 and hub 170 move into alignment. As shown in FIG. 14B, when the decoupling key 174 is engaged, the mecanum wheel 130 is coupled to the axle 154 and driven by motor 148. The decoupling system allows the wheel 130 to efficiently disengage from the drive axle 154 mitigating potential hazards associated with malfunctions or emergency situations.

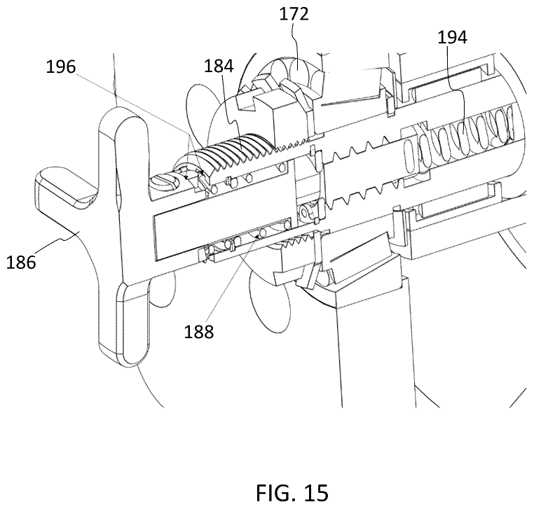

With further emphasis on FIG. 15, a self-locking, retained selector knob 186 may be used in the decoupling system as a safety feature to protect against inadvertent actuation. Similar to the decoupling key-hub interface, the selector knob and drive axle interface may have a set of interlocking teeth 196 to lock rotation of the selector knob 186. The selector knob 186 may include a two-piece assembly to facilitate assembly of the spring 188 and retaining ring into the axle shaft 154. In order to rotate the knob 186, the knob 186 may be pulled out and held axially while twisted in the desired direction. The preloaded return spring 188 biases the selector lever back to the locked position when released, thereby maintaining engagement of the wheel 130 to the drive axle 154 unless purposely released.

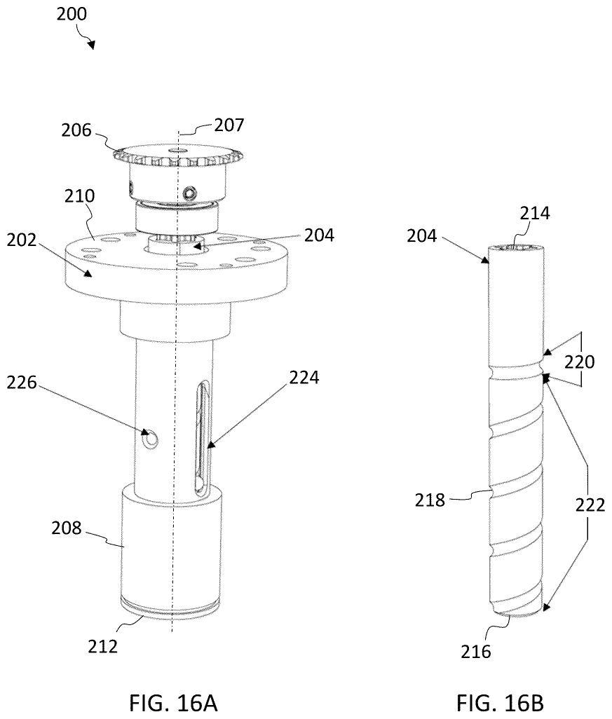

Turning now to FIGS. 16A-16B, a stabilizer assembly 200 is shown according to one embodiment. Stabilization may include the deployment of one or more rigid elements between the system base 12 and ground in order to restrict all relative motion between the base 12 and floor. In one embodiment, the stabilizer assembly 200 may include a rigid element that deploys from the base 12 and contacts the ground to prevent motion of the system 10 during procedures. The stabilizers 200 provide a more rigid link from the system to ground than is provided by just the casters 30 with brakes engaged. The surgical robotic system 10 may include one or more stabilizers 200 positioned, for example, at each wheel 30, at the front and back of the system 10, or in any suitable locations along the base 12 to stabilize the system 10. In one embodiment, front stabilizers 200A may be positioned within each front wheel assembly 30A and a rear stabilizer 200B may include a stabilizer module positioned centrally between the two rear wheels 30B of the base 12. The system rigidity immobilizes the robot's base 12 during operational tasks and ensures the accuracy of procedures utilizing the surgical robotic system 10, such as while performing musculoskeletal procedures.

The stabilizer assembly or module 200 may include an outer stationary housing 202, an inner stabilizer shaft 204, and a splined input shaft 206 aligned along a central axis 207. The stationary housing 202 may include a hollow base cylinder 208 for receiving the shaft and a mounting flange 210 for securing the stabilizer assembly 200 to the base 12 of the system 10. The stabilizer shaft 204 may include a post that is configured to protrude from the end 212 of the stationary housing 202. The stabilizer shaft 204 may include a drive recess 214 at its proximal end for transmitting torque to the shaft 204 from the splined input shaft 206. The stabilizer shaft 204 terminates at a distal tip 216 at its distal end, which is configured to engage the ground when deployed. The stabilizer shaft 204 defines a helical cut or groove 218, forming a spherical roller track to engage one or more ball bearings 224, 226, which guide deployment of the stabilizer shaft 204.

In one embodiment, the stabilizer module 200 may include a stabilizer shaft 204 with a variable helix 218. The variable helix configuration allows for rapid deployment of the stabilizer posts 204 with minimal mechanical input. The variable lead helix 218 may be provided to conserve motion by dividing the actuation into two stages: deployment and stabilization. The deployment portion or stage 220 of the helix 218 may have a large lead, low mechanical advantage to bring the stabilizer 200 from the retracted state down to the ground in 360° of rotation. The stabilization portion or stage 222 may be a low lead, high mechanical advantage which transfers load from the casters 30 to stabilizers 200 with minimal system movement over an additional 45°-90° of travel. The stabilization stage 222 may also have the added benefit of being non-back drivable due to the low helix angle, negating the need for additional motion constraint after deployment.

To achieve motion along the variable helix 218, an array of bearing balls 224 may be positioned between spherical undercut ball pockets in the stationary housing 202 and the spherical profiled, helical groove 218 in the rotating stabilizer. Due to the varying nature of the helix 218, a single constrained bearing ball 226 may be fully constrained in its pocket, which serves to transfer all load. The remaining bearing balls 224 may be unconstrained vertically and located in three equally spaced pockets. The free vertical ball bearings 224 may provide radial balancing forces to mitigate the stabilizer bowing under load from the fully constrained bearing ball 226. The ball bearing elements 224, 226 may help to keep friction low for improved efficiency. Although spherical ball bearings are exemplified, it will be appreciated that cylindrical roller bearings, tapered roller bearings, or other suitable bearings may be used.

The stabilizers 200 may be simultaneously linked to a motor 262 for automated deployment and a handle for manual override. The motorized deployment allows for rapid actuation of the stabilizer, while the manual override allows human operators to take direct control of the stabilizing functions. Due to the manual linkage, the actuation may be accomplished within the typical throw of a handle, rather than a multitude of turns from a direct-drive motor.

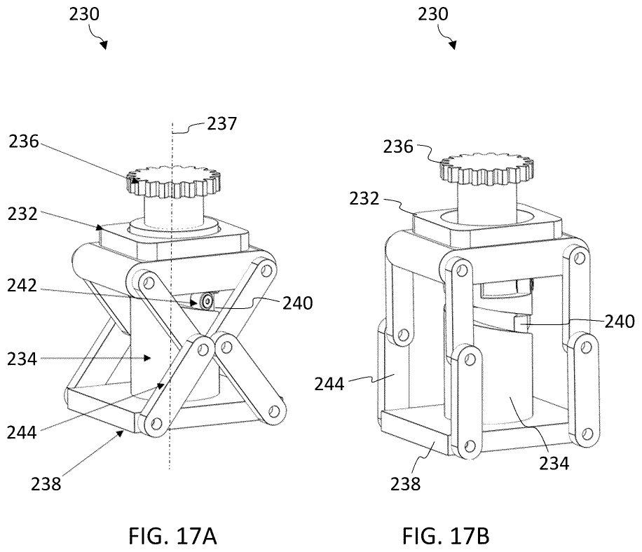

Turning now to FIGS. 17A-17B, an alternative stabilizer assembly 230 is shown. In this embodiment, a scissor linkage 244 aids in guiding deployment of the stabilizer assembly 230. The stabilizer assembly or module 230 may include a duplex bearing housing 232, an inner stabilizer shaft 234, a chain sprocket input shaft 236, and a stabilizer pad 238 aligned along a central axis 237. The stabilizer shaft 234 defines a helical cut or groove 240, forming a spherical roller track to engage one or more roller bearings 242, which guide deployment of the assembly 230. The input shaft 236 may include a sprocket or toothed wheel that engages with links of a chain, thereby forming part of a chain drive system for deploying the assembly 230. The input shaft 236 couples directly to the output shaft 234. A track roller 242 may be mounted to the input shaft 236 such that the track roller 242 rides in the helical track 240 in the output shaft 234. When the input shaft 236 is rotated, the track roller 242 follows the helical pattern in the output shaft 234 which results in linear travel. The duplex bearing housing 232 and stabilizer pad 238 may be coupled together via a scissor linkage 244. The scissor linkage 244 may include a plurality of arms or links pivotably coupled together, for example, with pins. In one embodiment, a linkage set of two pivotable links may extend from each corner of the assembly between the upper housing 232 and the lower stabilizer pad 238. The scissor linkage 244 anti-rotates the output shaft 234 and aids in guiding deployment. In FIG. 17A, the stabilizer assembly 230 is shown in a retracted position where the inner stabilizer shaft 234 retracts stabilizer pad 238 and the scissor linkage 244 is bent inward. In FIG. 17B, the stabilizer assembly 230 is shown in a deployed position where the inner stabilizer shaft 234 extends the stabilizer pad 238 and the scissor linkage 244 is arranged in parallel.

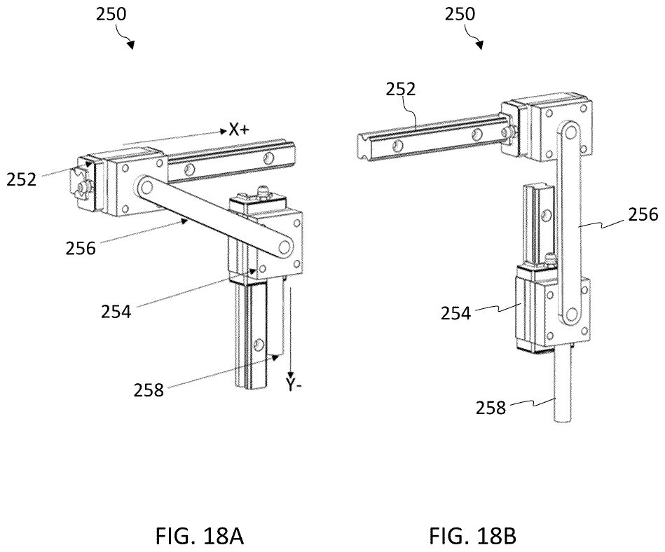

Turning now to FIGS. 18A-18B, an alternative stabilizer assembly 250 is shown. In this embodiment, connected carriages 252, 254 aid in guiding deployment of a stabilization foot 258, thereby immobilizing the robotic base 12. The stabilizer assembly or module 250 may include an input carriage 252, an output carriage 254, a linkage 256 between the carriages 252, 254, and a stabilization foot 258. The carriages 252, 254 may include linear ball slides for low-friction, constrained motion, for example. The stabilizer assembly 250 may include an over-center linkage 256 to achieve a mechanical advantage required for stabilization. The over-center linkage 256 uses pivot points positioned, such that, once moved beyond a certain angle, the linkage 256 locks into a vertical position, creating a stable position for the extended stabilization foot 258.

In one embodiment, the horizontal input carriage 252 and vertical output carriage 254 are connected via link 256 that couples their motion. When the horizontal carriage 252 moves X+, the vertical carriage 254 moves Y−. As the acute angle between the linkage 256 and sliders approaches 90°, the mechanical advantage of the input force over output force increases. Geometry may be sized such that the stabilization foot 258 contacts the ground when the linkage 256 is at an angle approaching 90°, with full stabilization being achieved at 90°. The portion of motion approaching and up to 90° drives the stabilizer foot 258 into the floor, removing load from the wheels 30, and stabilizing the system 10. With this motion occurring at a phase where the input has a high mechanical advantage over the output, stabilization may occur with input forces much lower than system weight.

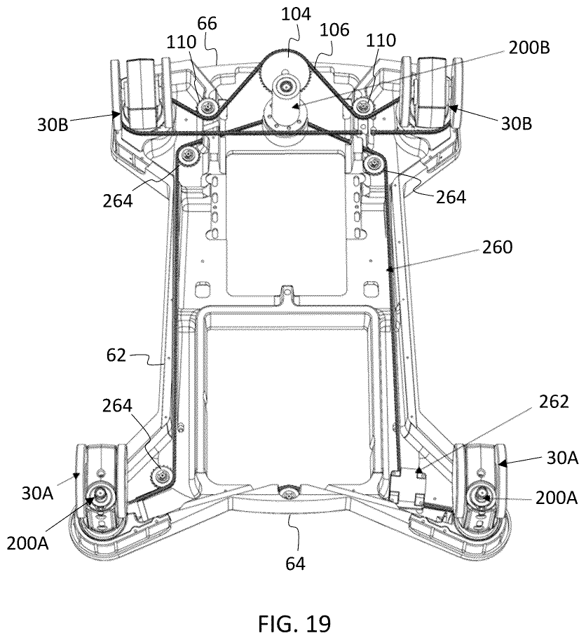

In one embodiment, the source of input for the input carriage 252 may include a stabilizer drive chain 260 coupled to the input carriage 252 and run to all stabilizers 250. The drive chain 260 may be combined with tensioners and idlers 264, as needed, to provide synchronized control. The chain 260 may be driven by a stabilizer deployment motor 262. Alternatively, individual actuators may be each coupled to a single stabilizer 252. In parallel with the individual actuators, a chain setup may be run for centralized, manual control. FIG. 19 depicts one example of a front and rear stabilization system for surgical robotic system 10. In this embodiment, three linked stabilizers 200A, 200B may be deployed to stabilize and immobilize the system 10. For example, left and right front stabilizers 200A may be located on the respective front wheels 30A, and a rear stabilizer 200B may be located between the rear wheels 30B. The front and rear stabilizers 200A, 200B may be linked via stabilizer drive chain 260, which is powered by motor 262. The linked configuration may allow for all stabilizers 200A, 200B to deploy simultaneously. Stabilizer deployment may be primarily accomplished with chain drive 260 to ensure synchronized movement. A manual override, for example, in the form of a lever attached to the chain 260, may be a secondary means of actuation or release of the stabilizers 200. Although front and rear stabilizers 200A, 200B are shown, it will be appreciated that any suitable type, combination, and placement of stabilizers may be selected for optimal immobilization. The stabilizers 200 may be deployed in order to achieve a statically determinant stabilization pose that is not susceptible to floor defects, such out-of-flatness or out-of-level conditions.

### Machine Vision End Effector

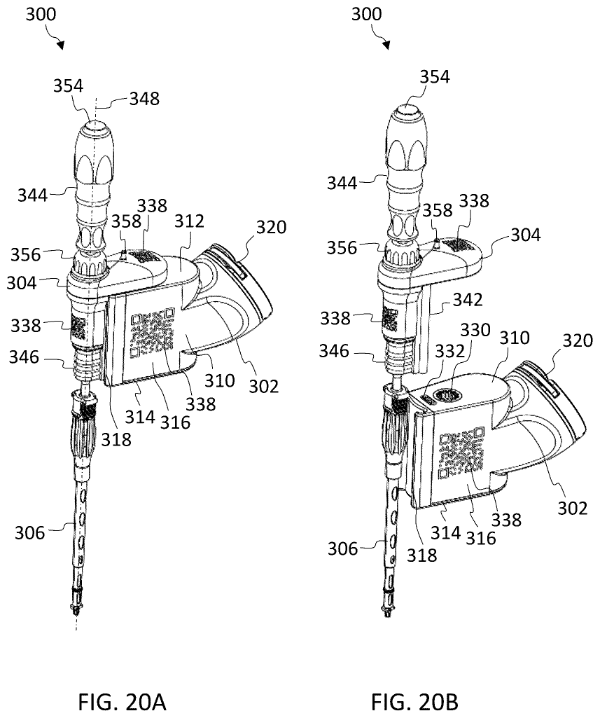

Turning now to FIGS. 20A-20B, an active machine vision end effector 300 is shown according to one embodiment. The end effector 300 is configured to couple to the end effector interface 42 of the surgical arm(s) 16. The end effector 300 may act as an extension of the surgical arm 16 and is configured to assist with navigated surgical robotic procedures. In one embodiment, the end effector 300 includes an end effector base 302, an instrument adaptor 304, and an instrument 306. The end effector 300 may be active, in that, the end effector 300 is powered and may provide rotary, oscillating, or other specialized motion to the instrument 306. The modular nature of the end effector base 302 and instrument adaptor 304 allows for many instrumentation options for robotically assisted procedures. The end effector 300 may also include machine vision markings 338 so that the machine vision camera 24 can determine the precise location of the end effector 300 and associated components in real time.

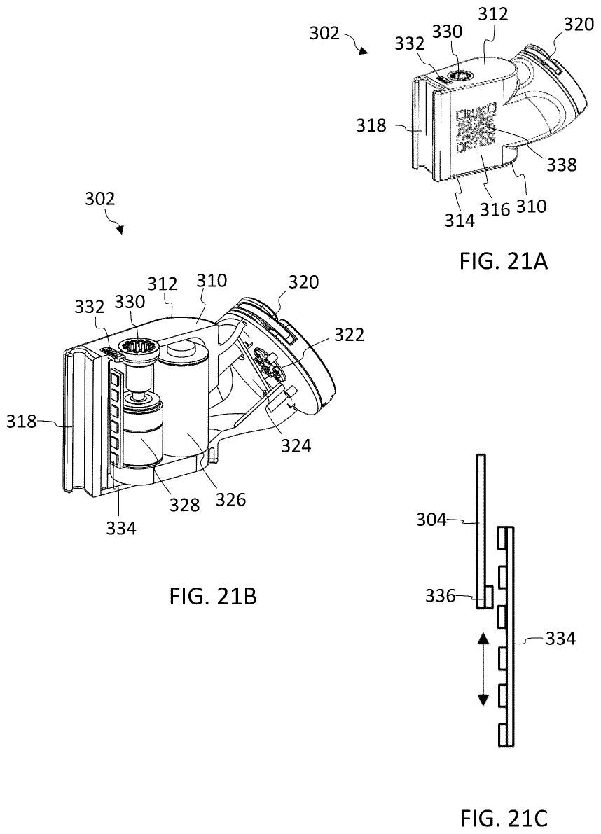

With further emphasis on FIG. 21A, the end effector base 302 may include a housing 310 having a top surface 312 and a bottom surface 314 connecting side surfaces 316. The front of housing 310 may define an adaptor rail track 318 configured for receiving and securing the instrument adaptor 304 therein. The back of the housing 310 may include a clamp interface 320 for attaching the end effector base 302 to the end effector interface 42 of the surgical arm 16. It will be appreciated that these relative positions are for descriptive purposes only as the base 302 may be reoriented in space when the end effector 300 moves with the end of the surgical arm 16.

The end effector base 302 serves as a sterile extension to the draped robotic arm 16. The end effector base 302 electrically connects and rigidly clamps to the robotic arm 16. For example, clamp 320 may be used to rigidly connect the end effector base 302 to the robotic arm 16. A connector 322 may be used to electrically connect to the robotic arm 16, providing both power and communication to the end effector 300. One example of a clamp for mechanically and electronically coupling a robot arm to an end effector is described in U.S. Pat. No. 11,684,437, which is incorporated by reference herein in its entirety for all purposes.

As best seen in FIG. 21B, the housing 310 holds a control board 324, a base motor 326, and a gearhead 328. The control board 324 is the brain of the end effector 300 providing bi-directional communication to the robotic arm 16, bi-directional communication to the instrument adaptor 304, motor control to the base motor 326, hall sensor monitoring, and power management to the instrument adaptor 304. The motor 326 and gearhead 328 are used to provide rotary motion to the instrument 306 with the required speed and torque for tasks such as drilling, tapping, or screw insertion. A power coupling 330 may be located on the top 312 of the housing 310, which mechanically connects the rotating shafts of the gearhead 328 to the instrument adaptor 304. A connector pad 332 may be used to transfer power and communication to the instrument adaptor 304. The connector pad 332 may be located on the top 312 of the housing 310 adjacent to the power coupling 330.

The adaptor rail track 318 is used as a mounting platform for the instrument adaptor 304. The adaptor rail track 318 may define a groove or recessed channel, which acts as a track or guide for the instrument adaptor 304. The rail track 318 may include two longitudinal ridges that flank the groove on either side. The rail track 318 may be linear, curved, or contoured along its length. The instrument adaptor 304 may slide along the groove and seat within the adaptor rail track 318. The adaptor rail track 318 may be used to guide the instrument adaptor 304 along the linear trajectory held by the robot 10. The adaptor rail track 318 may provide for two types of control for the surgeon: guidance and assistance. The first is guidance as the robot arm 16 moves to a trajectory and the user controls the depth of the instrument 306 along the linear trajectory. The second is assistance as the instrument adaptor 304 is securely attached to the end effector base 302 and only active motion is permitted.

A hall sensor array 334 may be used to determine the location of the instrument adaptor 304 as it travels along the adaptor rail track 318. The hall sensor array 334 may include a plurality of hall sensors arranged, for example, in a linear pattern along the length of the adaptor rail track 318. As best seen in FIG. 21C, a magnet 336 may be attached to the instrument adaptor 304, which may be sensed by the hall sensor array 334. As the instrument adaptor 304 is positioned along the adaptor rail track 318, the hall sensor array 334 detects the location of the instrument adaptor 304. For example, using the proportional signal strength of each hall sensor, the position of the instrument adaptor 304 along the adaptor rail track 318 can be determined by the system 10.

Machine vision cameras 24 may be used to track the end effector 300, for example, using visual markings and geometric information of the end effector 300. Tracking the end effector 300 directly by the camera 24 helps to remove errors in the forward kinematics of the robotic arm 16. The end effector base 302 may include machine vision markings 338 so that the machine vision camera 24 can determine the precise location of the end effector 300 in real time. Unlike end effectors using infrared LEDs which have a limited life cycle due to the autoclave process, end effector 300 may include machine vision markings 338, which hold up repeatedly under the harsh autoclave conditions. The machine vision markings 338 may include optical, machine-readable representations of data, such as barcodes, data matrix, quick-response (QR) codes, Aztec codes, DotCodes, or the like. In one embodiment, the machine vision markings 338 include QR codes. The machine vision markings 338 may be located on any suitable locations on the housing 310 of the end effector 300. For example, machine vision markings 338 may be located on one or both sides 316 of the housing 310. The markings 338 may also provide improved navigation by supporting machine vision, which may be used by the machine vision camera 24 to determine the precise location of the end effector 300 in real time.

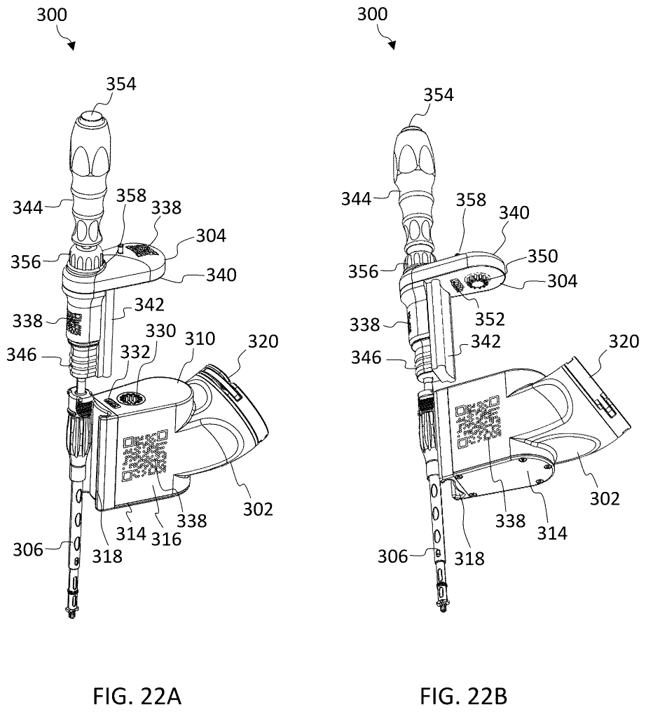

With further emphasis on FIGS. 22A-22B, the instrument adaptor 304 serves as an interface between the instrument 306 and the end effector base 302. The instrument adaptor 304 electrically connects and rigidly clamps to the end effector base 302 and is designed to interface with many types of instruments 306, such as drills, burrs, taps, screwdrivers, interbody holders, and rod holders, to name a few. The instrument adaptor 304 allows for top-down instrument attachment, which may help to improve the surgeon's workflow.

The instrument adaptor 304 may include a casing 340, an adaptor rail 342, a handle 344, and a quick connector 346. The casing 340 is configured to engage with the end effector base 302. The instrument adaptor 304 allows for top down instrument attachment which may enhance the surgeon's workflow. The adaptor rail 342 extends from the casing 340 and may include a linear rail sized and dimensioned to fit within the corresponding adaptor rail track 318 on the end effector base 302. The rail 342 may be curved or contoured to fit the corresponding track 318. The adaptor rail 342 may be configured to slidably mate with the corresponding adaptor rail track 318. The adaptor rail 342 may be used to guide the instrument adaptor 304 along the linear trajectory held by the robot 10 to provide guidance and assistance control for the surgeon. For guidance control, the robot 10 moves to a trajectory and the user controls the depth of the instrument 306 along the linear trajectory. For assistance control, the instrument adaptor 304 is securely attached to the end effector base 302 and only active motion is permitted.

The handle 344 may include a grip configured to be grasped by the user. The handle 344 may be used by the user to manually rotate the instrument shaft 306. The quick connector 346 may include a quick connect fitting used to provide a fast connection to attach the instrument 306. A sensor may be used to determine if an instrument 306 is attached to the quick connect 346. The handle 344 and instrument 306 may be aligned along a tool axis 348, which follows the trajectory determined by the robotic system 10. The quick connect 346 may be used to allow the user to quickly switch instruments 306. Instruments 306 may include drills, burrs, taps, screwdrivers, or other instruments for inserting screws, for example. Instruments 306 may include rod holders and inserters, rod pushers, rod reducers, or other instruments for installing spinal rods, for example. The instruments 306 may further include dilators, disc preparation instruments (e.g., curettes, Cobb elevators, osteotomes, rasps, scrapers, etc.), trials, retractors/distractors, inserters, interbody holders, and other instruments for installing interbody implants, for example. It will be appreciated that any suitable instruments may be used for the designated surgical procedure.

The instrument adaptor 304 may be mechanically and electronically coupled to the end effector base 302. The power coupling 350 is used to mechanically connect the rotating shafts of the end effector base gearhead 328 to the instrument adaptor 304. The control board and electronic connector 352 is configured to connect with connector pad 332. The control board and connector 352 may be configured to transfer power and communication from the end effector base 302, motor control to the instrument adaptor motor 326, determine instrument attachment state, determine direction control state, determine button press position, and measure force and torque. The power coupling 350 and connector 352 may be located on an underside of the casing 340 such that when the adaptor rail 342 fully slides down track 318 and the casing 340 contacts the top surface 310 of the end effector base 302, the adaptor 304 is fully connected to the end effector base 302.

The top of the handle 344 may include a drive button 354, which may be pressed by the user to rotate or otherwise control the instrument 306. The drive button 354 may be used to mechanically engage the instrument shaft 306 with the motor drive 326. When depressed, a proportional signal may be sent to the control board 324 to rotate the motor 326 at the desired speed. A direction control knob 356 may be located on the top of the casing 340 at the base of the handle 344. The direction control knob 356 may be used to set forward ratchet, reverse ratchet, and shaft lock, for example.

Similar to the end effector base 302, one or more machine vision markings 338 may be located on the instrument adaptor 304 and used by the machine vision camera 24 to determine the precise location of the instrument 306 in real time. For example, a machine vision marking 338 may be located on the top of the casing 340 and on an outer face above the quick connect 346. Machine vision markings 338 may be located in any suitable locations on the housing of the instrument adaptor 304. Visual markings 338 may be placed on the instrument adaptor 304 to allow the adaptor 304 to be tracked independently. The machine vision marking 338 on the instrument adaptor 304, or in different locations, may be different than machine vision markings 338 on the end effector base 302 so that the system 10 can distinguish between the two components or locations. The instrument adaptor 304 may also include a neuro monitoring post 358, for example, on the top of the casing 340, which is used to connect neuro monitor leads to the instrument 306.

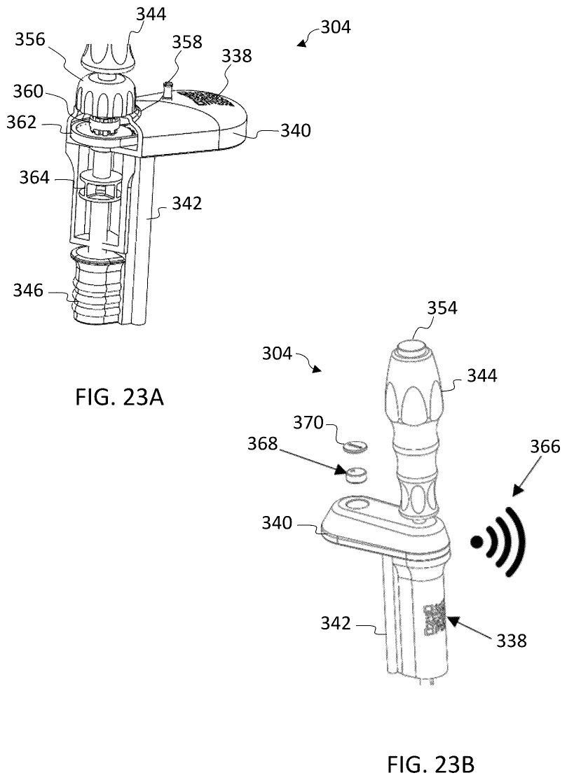

With further emphasis on FIG. 23A, the internals of the instrument adaptor 304 may include a drive clutch 360, a drive 362, and a force and torque sensor 364. The drive clutch 360 may be used to engage and disengage the instrument shaft 306 from the motor drive 362. The drive clutch 360 may provide a natural feel when the user uses the handle 344 in manual mode without feeling the drag of the motor drive 362. The drive 362 may be used to rotate the instrument shaft 306 by the motor 326. The force and torque sensor 364 may be used to measure axial force along the instrument shaft 306 and torque about the instrument shaft 306. By obtaining information about torque and axial force, the system 10 may perform automated tapping and screw insertion. The force and torque sensor 364 may also allow the system 10 to determine if the bone is breached based on a comparison to a normal loading profile.

With further emphasis on FIG. 23B, the end effector 300 may be configured for intelligent navigation. The instrument adaptor 304 may provide continuous communication to the robot 10 during the procedure. This may be accomplished via machine vision navigation coupled with wireless communication 366. The casing 340 may define an opening for receiving a battery 368 and battery cover 370 for securing the battery 368. The battery 368 may be configured to power a wireless transmitter and/or receiver 366.

According to one embodiment, an intelligent navigation workflow may include one or more of the following steps. First, inserting the battery 368 into the instrument adaptor 304. Second, inserting an instrument 306 into the instrument adaptor 304. Third, holding the instrument adaptor 304 toward the machine vision camera 24. The machine vision markings 338 may provide both a unique serial number of the instrument adaptor 304 as well as positional information needed for navigation. Fourth, the machine vision camera 24 may automatically perform identification and verifications steps including determining which instrument 306 is attached, calibrating the instrument tip location and trajectory, and verifying that the correct implant is attached. Fifth, the robot 10 can connect to the instrument adaptor 304 via a wireless protocol, such as Wi-Fi or Bluetooth Low Energy (BLE). By knowing the unique serial number of the machine vision markings 338, the robot 10 may automatically connect to the instrument adaptor 304. The wireless communication 366 allows the instrument 306 to be used attached or detached from the robot 10. The wireless communication 366 also ensures that the system 10 knows if an instrument 306 is removed and/or replaced with another.

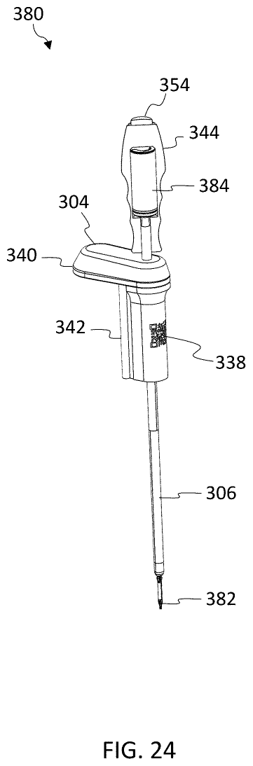

Turning now to FIG. 24, an oscillating drill adaptor 380 is shown according to one embodiment. In this embodiment, the instrument 306 may be a high-speed drill, burr, or cutting tool, for example, with a drill end 382 configured for drilling, milling, and/or tapping procedures. In these types of applications, an additional motor 384 may be placed in the handle 344 or body of the instrument adaptor 304. The motor 384 may be configured to provide rotational or oscillating motion to the drill end 382. The additional motor 384 allows the motor 326 in the end effector base 302 to be optimized for high-torque/low-speed applications, while the motor 384 in the handle 344 is optimized for high-speed/low-torque applications. Putting the motor 384 into the instrument adaptor handle 344 removes the inertia from the motor drive which will allow for improved operation. Minimizing the drill inertia is useful for oscillating drilling and milling applications due to the high-frequency back and forth motion.

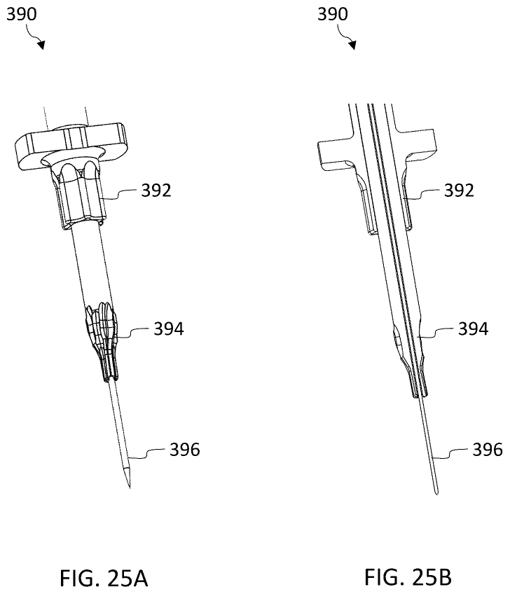

Turning now to FIGS. 25A-25B, a combo drill and screwdriver adaptor 390 is shown according to one embodiment. The flexible design of the end effector base 302 and instrument adaptor 304 allows for many functional combinations. In this embodiment, screw-driving and drilling are combined into one setup. The combo drill and screwdriver adaptor 390 may utilize a canulated screwdriver 392 that is driven by the motor 326 in the end effector base 302 combined with the oscillating drill instrument adaptor 380 shown in FIG. 24. In this embodiment, the drill tip 394 is positioned through the screwdriver 392. The drill may be cannulated to receive a K-wire or guide wire 396. The oscillating drill tip 394 may be advanced when the screwdriver 392 is rotated in the reverse direction. For example, rotating the screwdriver 392 in the forward direction may retract the drill tip 394. A self-tapping screw may be used to allow for a single step screw insertion.

The drill and screwdriver workflow may include one or more of the following steps. First, the robot arm 16 is moved onto the desired trajectory and advanced down to the surface of the bone. Second, the high-speed oscillating drill 394 is turned on. Third, the screwdriver 392 is rotated in the reverse direction until the drill 394 is plunged into the bone to the desired depth. Fourth, the robot arm 16 moves down and the screwdriver 392 begins to rotate in the forward direction. The forward screw rotation begins to retract the drill 394 so it does not advance further into the bone. The speed of the robot arm 16 and screwdriver rotation speed may be synchronized, for example, due to (1) knowledge of the screw pitch, such as rigid tapping in CNC operations; and/or (2) using the axial force meter in the instrument adaptor 304 to maintain a constant preload can ensure the screw is driven into place, which is similar to a manual screw insertion.

In one embodiment, the instrument adaptor 304 may include a jack hammer adaptor or tip. The jack hammer may be characterized by rapid, repetitive movements and/or vibratory forces to drive the tip back and forth against the bone surface. For example, motor 384 may impart the rapid hammering motion to the adaptor tip. The jack hammer mechanism may allow for interbody placement and osteotomies without the need for large amplitude hammer swings. A low amplitude/high frequency vibration reduces the force to the patient while also providing added safety. Breaking through can sometimes cause large overshoots in positions and in some cases could cause harm to the patient, which could result in spinal cord and nerve damage or vasculature breach.

In another embodiment, the instrument adaptor 304 may include an arthroplasty sagittal saw adaptor or tip. The hardwired power and communication allow for other end effector designs, such as a sagittal saw used in total knee arthroplasty (TKA) applications. The motor 384 may provide for variable speed controls, allowing for cutting speed adjustments based on the tissue or bone being cut. The robot arm 16 may position the saw based on patient anatomy and the saw may be powered on and off by the system 10 and/or the surgeon as required by the procedure. It will be appreciated that other suitable adaptor tips may be selected based on the surgical procedure.

Prior to use, the sterility of the end effector 300 may be achieved using an autoclave process. The machine vision end effector 300 may be configured to have autoclavable longevity such that the effector 300 lasts for many cycles in harsh autoclave conditions. Unlike other end effectors that may have a limited life, for example, due to integrated infrared LEDs, these trackers may be replaced with physical markings 338 that are trackable by the machine vision cameras 24. Other protective measures may also include seals at all patient contacting interfaces to ensure proper biocompatibility, a conformal coating applied to the printed circuit boards (PCBs) to protect against moisture, ensuring all components are rated for autoclave temperatures, and providing a vent at the clamping interface, away from the patient, to allow all moisture to exit during the autoclaving. The autoclave process ensures the end effector 300 is sterilized and free from contaminants before coming into contact with the patient, thereby maintaining patient safety.

Sterile Drape with Electrical Connectivity

When performing surgery and dealing with exposed internal tissues, it is necessary to minimize the risks of introducing harmful bacteria, viruses, and fungi into the surgical site. This reduces the chances of post-operative infections. When done properly, surgical sterilization considerably minimizes the chances of cross contamination between the patient, medical professionals, and equipment, further creating a safe and productive surgical environment. A sterile drape may be used to ensure and continue the practice of surgical sterilization to allow for optimal patient safety and healing. Medical drapes may be used during surgery to create a sterile barrier and maintain an uncontaminated field around the surgical site. Medical drapes may be used on patients and to cover any technology that is unable to undergo the proper sterilization methods.

The end effector may attach to the robotic arm on top of the sterile drape. In some cases, there may be a little play between the arm and end effector connection in order to avoid any tears or rips in the drape, which may result in some inaccuracies. In addition, the amount of power and data transmitted through the drape may be limited because there is not a direct connection. The limited power of the end effector may prevent the end effector from performing high power activities, such as drilling or milling. When transmitting power and data through a sterile drape, wireless transfer or direct connections may be used. For wireless technologies, such as inductive coupling or radio frequency (RF) transmission, the wireless transmission allows a continuous sterile barrier to be maintained, but the amount of power and data transmitted may be limited. For direct connections, for example, through flaps or windows in the drape, without a completely sealed interface, these openings in the drape allow for the cables and devices to make direct electrical connections, but they also allow for possible contamination of the sterile field.

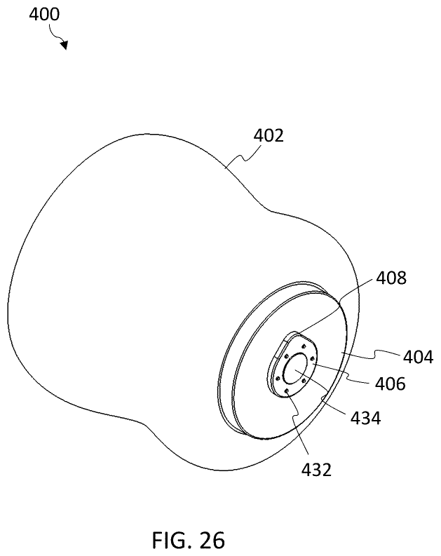

Turning now to FIGS. 26-29B, a sterile drape assembly 400 is shown according to one embodiment, which preserves sterility and electrical connectivity. This allows the end effector to remain sterile while receiving direct power and data across the sterile barrier. The sterile drape assembly 400 may provide a rigid connection between the end effector, such as active end effector 300, and robotic arm 16 while ensuring the sterile field is not compromised. This removes all play from the connection point and thus improves the accuracy of the system 10. The continuous sterile barrier allows for electrical cable connection without the need for an unsealed hole in the drape. The assembly 400 provides for highly efficient power transmission, without heat generation, as opposed to wireless power transfer, which can generate significant heat. The assembly 400 provides simple and robust data transmission via a physical connection, as opposed to wireless connectivity, which is prone to complexity of establishing connectivity and intermittent data transfer.

With reference to FIG. 26, a sterile drape assembly 400 may include two parts, a sterile drape 402 and a connector cap 404 attached to the sterile drape 402. The sterile drape 402 may be made from a sterile material, such as film or nonwoven fabric, that is designed to resist penetration by liquids and microbial agents, ensuring that the area beneath it remains sterile. The cap 404 may be welded or otherwise secured to the drape 402. The cap 404 may include a plastic cap, for example, having a disc shape sized and dimensioned to interface with the end effector interface 42 on the robot arm 16 and the clamp 320 of the end effector 300. The plastic cap 404 retains an electrical connector 406, which transmits power and data from the robot arm 16 to the end effector 300. The electrical connector 406 may be flush, inset, or raised relative to the cap 404. In one embodiment, the connector 406 may have a raised disc shape with an embossed or asymmetrical edge 408, which orients the plastic cap 404.

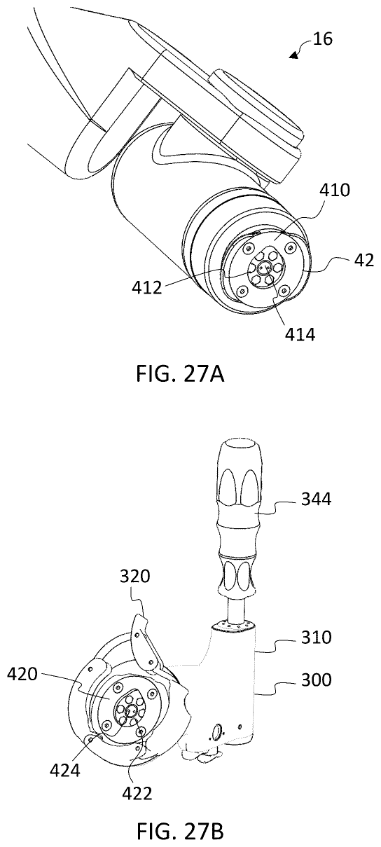

The process of transmitting power and data through the robotic arm 16 to end effector 300 begins at the palm assembly or end effector interface 42 and ends at the end effector 300. With reference to FIG. 27A, the distal end of robot arm 16 may include end effector interface 42 having a mounting flange 410 with a conducive pad 412 and a ferrous target 414. The conducive pad 414 may be located centrally within or on the mounting flange 410 and the ferrous target 414 may be located at the center of the mounting flange 410. With reference to FIG. 27B, the clamp 320 of the end effector 300 may include a similar mounting flange 420 with conducive pad 422 and ferrous target 424. Both the palm assembly 42 and end effector 300 include electrically conductive pads 412, 422 that facilitate connectivity through the sterile drape assembly 400. The palm assembly 42 and end effector also include ferrous targets 414, 424 that allow for a magnetic connection.

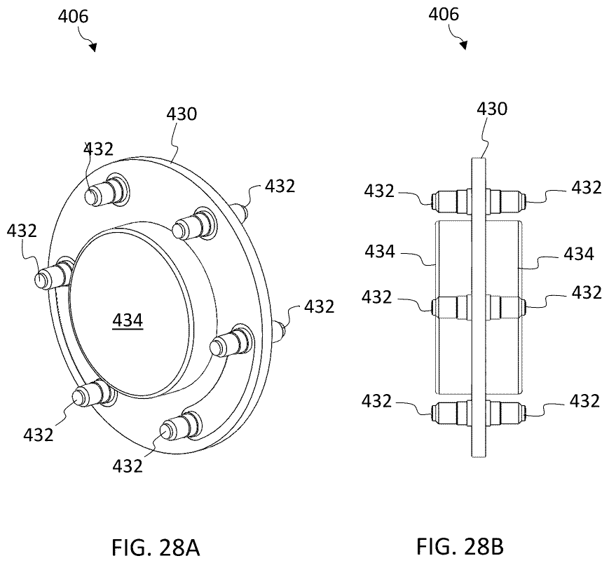

With further emphasis on FIGS. 28A-28B, the plastic cap 404 encapsulates a printed circuit board (PCB) 430 which has pogo pins 432 and a magnet 434 on each side. The pogo pins 432 or spring-loaded pins are a type of electrical contact used to establish a connection between two circuit points. The pins 432 may include a pin, a spring, and a barrel such that the pin moves within the barrel under the force of the spring for a reliable electrical connection. The corresponding pogo pins 432 are electrically connected from one side to the other. The first set of pogo pins 432 align with the conductive pad 412 connected to the robotic arm 16. The embossed or raised edge 408 found within the sterile plastic cap 404 facilitates proper alignment when attaching the sterile cap 404 to the palm assembly 42 and end effector 300 through their corresponding indents. When attaching the sterile drape 400, the plastic cap 404 is positioned around the mounting flange 410.

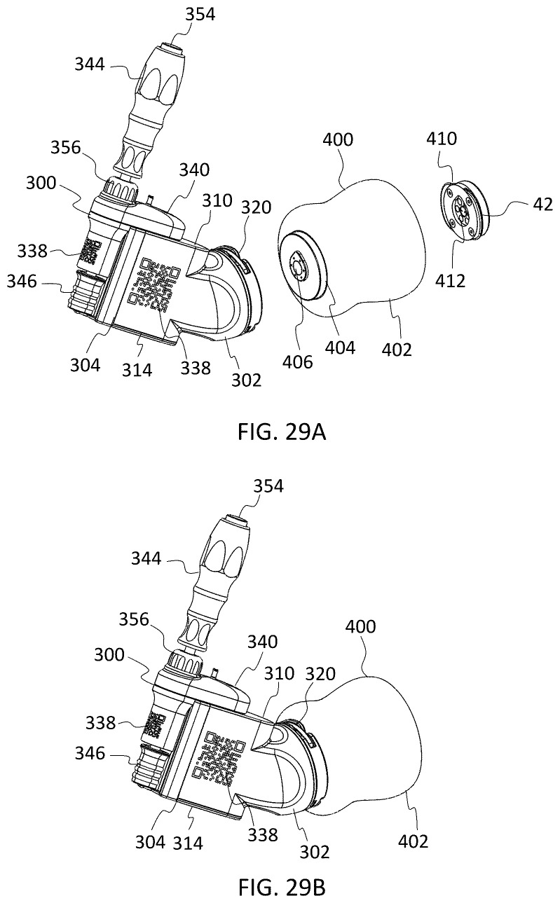

As best seen in FIGS. 29A-29B, the sterile drape assembly 400 is positioned between the mounting flange 410 of the robot arm 16 and the clamp 320 of the end effector 300. The magnet 434 attached to the inside of the sterile cap 404 connects to the palm assembly 42 holding the sterile cap 404 in place before the end effector 300 is connected. When the end effector 300 is positioned and clamped, the plastic of the sterile cap 404 forms around the mounting flange 410 removing all play and creates a rigid connection. This further enhances accuracy and removes any possible damage to the drape 402 in the process. Without the plastic cap 404, the drape 402 could be susceptible to tearing, which would result in the contamination of the sterile field. During the end effector attachment, the second set of pogo pins 432 align with the conductive pad 422 connected to the end effector 300. In this manner, the corresponding pogo pins 432 align with the respective conductive pads 412, 422, thereby completing the electrical connectivity. Due to the plastic cap 404 and electrical connectivity built into the drape assembly 400 itself, flaps and windows are not necessary. Instead, the sterile drape assembly 400 allows the end effector 300 to remain sterile while receiving direct power and data from the robot arm 16.

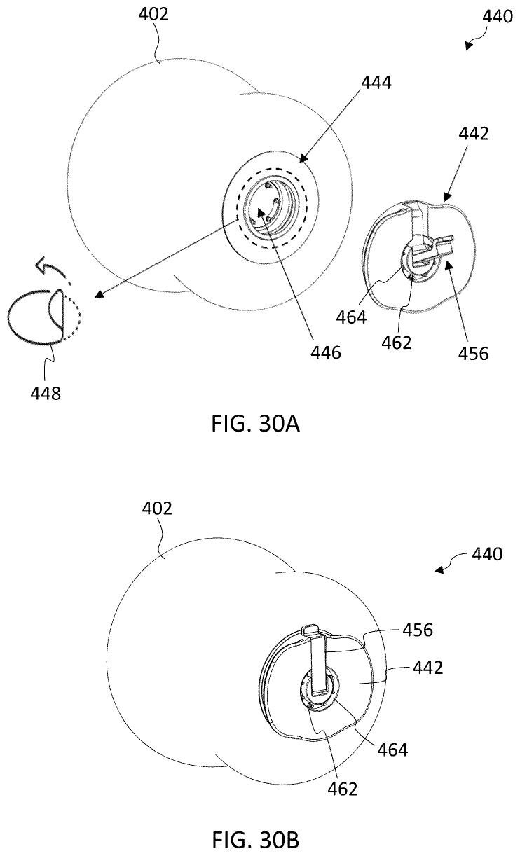

Turning now to FIGS. 30A-30B, a sterile drape assembly 440 is shown according to another embodiment, which preserves electrical connectivity. In this embodiment, an alternate way to transmit power and data across the sterile field is with the use of a sterile, sealed intermediate clamp 442. The sterile drape assembly 440 has an opening in the drape 402, which is sealed by attaching the intermediate clamp 442.

The sterile drape assembly 440 may include sterile drape 402 attached to a ring 444. Similar to cap 404, the ring 444 may be welded or otherwise secured to the drape 402. The ring 444 may include a plastic ring, for example, having a loop-like shape sized and dimensioned to interface with the end effector interface 42 on the robot arm 16 and the intermediate clamp 442. The plastic ring 444 retains an electrical connector 446, for example, having the printed circuit board 430 and pogo pins 432, which transmit power and data from the robot arm 16 to the intermediate clamp 442 and ultimately to the end effector 300. The electrical connector 446 may be inset relative to the ring 444. The opening or annulus of the plastic ring 444 may be sealed with a removable adhesive cover 448. The cover 448 maintains sterility, while the drape 402 is installed over the arm 16. Once the intermediate clamp 442 is ready to be attached, the adhesive cover 448 is removed, the intermediate clamp 442 is inserted into the arm 16, and the electrical connections are made.

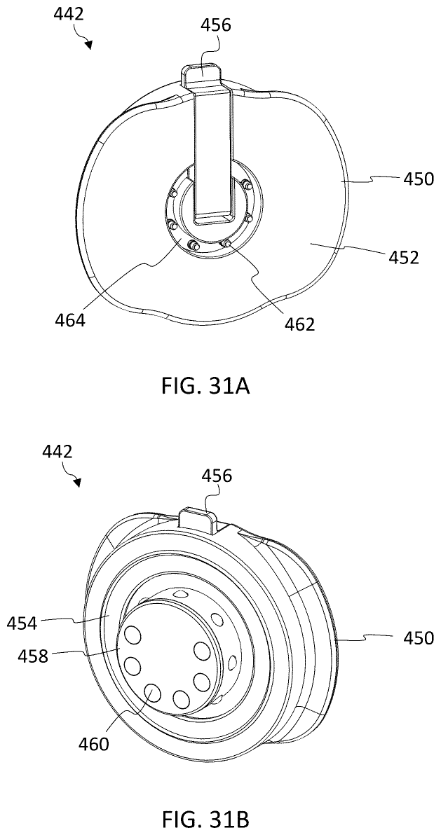

As best seen in FIGS. 31A-31B, the intermediate clamp 442 may include a plug 450 with a front surface 452 for contacting the end effector 300 and a back surface 454 for contacting the plastic ring 444 and the electrical connector 446. The intermediate clamp 442 may include a locking mechanism, for example, actuated by a lever 456, which attaches to the end of the robotic arm 16 and seals the clamp 442 against the plastic ring 444. In one embodiment, the front surface 452 of the plug 450 may define a lever 456, which is configured to seal the intermediate clamp 442 to the plastic ring 444. The lever 456 may include a hinged tab that fits into a recessed groove on the front surface 452 of the plug 450. The back surface 452 of the intermediate clamp 442 may include a protruding cylinder 458 sized and dimensioned to fit in the corresponding annulus of the ring 444 to establish a connection. Power and data may be transmitted through the intermediate clamp 442 via conductive pads 460 on the back and pogo pins 462 at the front. The conductive pads 460 may be directly attached to the base of the pogo pins 462 such that they are continuous pins from back to front. Alternatively, the conductive pads 460 may be placed on a printed circuit board (PCB), which the pins 462 are soldered to on the other side. The pogo pins 462 may be located in an annular groove 464 defined into the front surface 452 around the hinged portion of the lever 456.

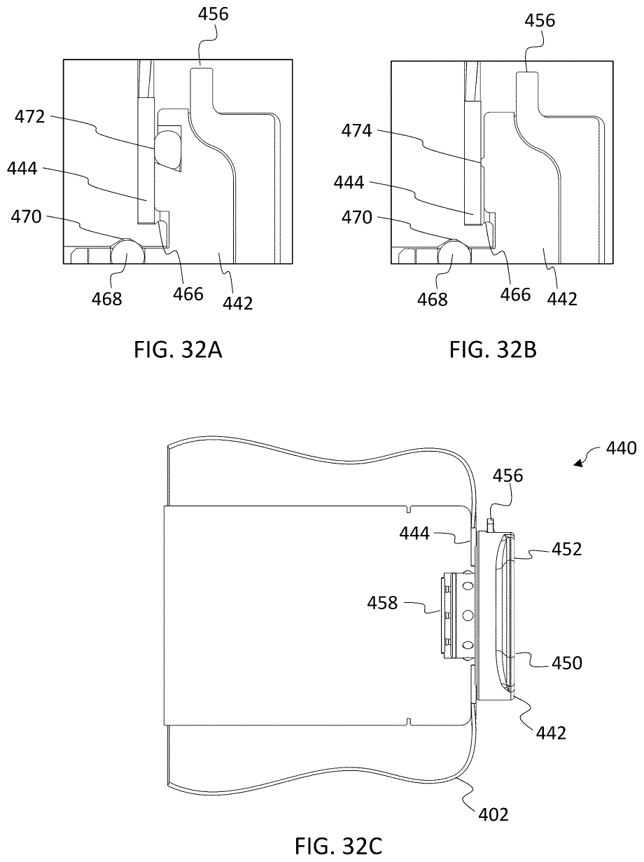

As best seen in FIGS. 32A-32C, during installation of the drape 402, the plastic ring 444 may be snapped in place over a notch 466 at the end of the robotic arm 16, which securely holds the ring 444 in place before the intermediate clamp 442 is attached. The intermediate clamp 442 may be sealed to the ring 444 and robotic arm 16 using one or more ball bearings 468 which lock into a corresponding groove 470 in the arm 16. After the intermediate clamp 442 is inserted into the end of the robotic arm 16, the lever 456 may be actuated to expand the ball bearings 468 outward, locking into the corresponding groove 470 within the robotic arm 16. This causes the intermediate clamp 442 to be pulled back against the plastic ring 444, creating a seal while maintaining a rigid mechanical connection.

The intermediate clamp 442 may also seal against the plastic ring 444 welded to the drape 402, for example, using an O-ring seal 472 or metal seal 474. As shown in FIG. 32A, the O-ring seal 472 may be stretched over a half-dovetail groove to keep the O-ring 472 in place when the intermediate clamp 442 is not attached. When the intermediate clamp 442 is securely attached to the end of the robotic arm 16, the O-ring 472 is pressed against the plastic ring 444 and the non-sterile opening at the end of the robotic arm 16 is contained. Alternatively, as shown in FIG. 32B, the metal seal 474 may include a boss on the clamp surface, for example, with a triangular cross section that bites into the plastic ring 444 when clamped down. It will be appreciated that any suitable mechanism for sealing the intermediate clamp 442 including gaskets, compression seals, lip seals, etc., against the plastic ring 444 may be used to maintain sterility and ensure the integrity of the connections. Once the intermediate clamp 442 is attached, the opening in the drape 402 is sealed, thereby maintaining a sterile field throughout the procedure while receiving direct power and data across the sterile barrier.

### Ultrasound Tracking

In addition to or as an alternative to optical-based tracking, for example, of active or passive markers or machine vision markings, ultrasound-based tracking may be utilized by the system 10. Ultrasound utilizes high-frequency sound waves, not audible to human hearing, which monitor the position and movement of internal body structures in real time. The ultrasound device may include an ultrasound (US) transducer, for example, attachable to the robot arm 16, attachable to the patient, or used freely in the operating space, which provide real-time images to the robot system 10. Details on examples of US transducers and ultrasound tracking are further described in U.S. Pat. No. 11,850,009, which is incorporated by reference herein in its entirety for all purposes. Ultrasound tracking may be used, for example, to: (1) automatically register the patient anatomy to optical tracking without irradiating the patient; (2) simultaneously register ultrasound to bone and optical tracking to ultrasound, providing registered optical tracking without requiring incision; and/or (3) allow multiple ultrasound devices to work together, providing tracking of deformity correction or other surgical or non-invasive maneuvers. Ultrasound registration allows registration without exposing the patient to ionizing radiation and a registration process that is completed more quickly than standard preop CT (computed tomography) registration. Ultrasound-enhanced tracking allows patient tracking without requiring an incision. Besides being less invasive, ultrasound-enhanced tracking may allow operating room support staff to complete registration before the surgeon enters the room. Ultrasound guidance may also improve accuracy and remove concerns over line of sight issues.

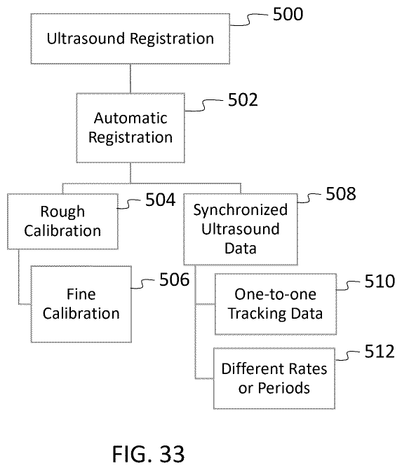

Turning now to FIG. 33, ultrasound registration 500 may be used to register the patient anatomy to optical tracking. Unlike registration systems utilizing X-rays or intraoperative CT scans, ultrasound registration 500 does not require ionizing radiation of the patient. Ultrasound registration 500 may work with CT or MRI (magnetic resonance imaging) and may register optical tracking to the scan by matching bony surfaces detected by the tracked ultrasound sensor with corresponding surfaces in the scan. To perform such a registration 500, the user may move a tracked ultrasound probe over the anatomy of interest until a match of the bony surfaces is achieved. During ultrasound probing, the optically tracked markers on the probe are visible to the cameras 24 as well as the optically tracked markers on the patient reference array (a.k.a., dynamic reference base or DRB).

In one embodiment, the ultrasound registration process may be accomplished automatically by the robot. The automatic registration 502 may include a rough calibration 504 and a fine calibration 506. After the DRB has been attached, the rough calibration 504 of the patient to the optical tracking system may be established that informs the system 10 approximately where to start looking for registration. The ultrasound transducer or probe may be attached to the robot arm 16. The robot 10 then automatically moves the optically tracked ultrasound probe into place and starts sweeping back and forth, looking for matching anatomy. Robotically controlling this procedure allows the robot 10 to automatically change its grip and arm position to optimize line of sight to the cameras 24 and ensure that the DRB remains visible. After registration is achieved, the rough calibration 504 may be replaced with fine calibration 506. The fine calibration 506 may include a more focused and detailed sweep of the surgical area to optimize image quality and measurement accuracy.

The rough calibration 504 may only need to be accurate to a few centimeters but allows automatic revision. One way of establishing the rough calibration 504 may be to have the user point with an optically tracked probe to points on the patient where some visually prominent landmarks are, such as the hip, tailbone, base of neck, or spinous processes. Another way of establishing the rough calibration 504 may be through a shape model of the patient, where the visible light image received by the tracking cameras 24 (e.g., in the coordinate system of the tracking cameras) is fed to a shape model, which predicts what the blobs of the image correspond to (e.g., legs, arms, back, head, spine) and then establishes roughly where the surgical anatomy is located in the camera's coordinate system.

In one embodiment, frames of tracking data (optical tracking arrays on the ultrasound probe and DRB) may be synchronized with ultrasound data 508. Synchronization 508 may be one-to-one 510 (e.g., every frame of tracking data has a corresponding frame of ultrasound data), or data from the different streams may be received at different rates or periods 512 and matched based on timers or other triggers. From the two data sources, registration 500 may be processed and a transformation of coordinates from CT to DRB coordinate system computed. Each additional frame of ultrasound+tracking data may be used to recompute the CT to DRB coordinate transformation and averaged with previous frames. The process may be continued until a threshold number of frames has been used for computing registration of a particular bone and the standard deviation of the mean transformation is within an allowable threshold. In one embodiment, frames of tracking and ultrasound data may be collected at 1 Hz. Progress of registration 500 may be conveyed to the user, for example, with a progress bar that goes from zero to full size as frames are accumulated. In one embodiment, each vertebra being registered may have its own progress bar and the user may continue to move the ultrasound probe until all levels are successfully registered and progress bars are at full size. It will be appreciated that other suitable indicators may be given to the user to designate progress and completion of the registration 500.

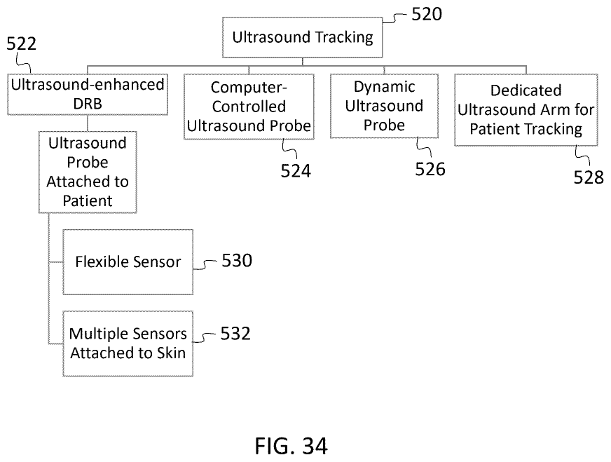

Turning now to FIG. 34, ultrasound patient tracking 520 may be used with robotic system 10, for example, for tracking a patient during the surgical procedure. In one embodiment, ultrasound patient tracking 520 includes an ultrasound-enhanced dynamic reference base (DRB) 522, which mounts to the skin of the patient instead of being anchored to bone with screws, spikes, or clamps. The ultrasound-enhanced DRB 522 is able to track movement of the bone relative to the cameras 24 using ultrasound to track skin-to-bone movement and optical tracking to track camera-to-skin movement. Ultrasound tracking 520 may further include: (1) an automatic computer-controlled ultrasound probe 524 to intelligently guide the ultrasound imaging; (2) a dynamic ultrasound probe 526 to continuously and systematically scan targeted areas; (3) a dedicated ultrasound arm for patient tracking 528; (4) a flexible multi-part ultrasound sensor 530 configured to track the patient; and (5) multiple ultrasound sensors 532 attached to the skin of the patient, during or after the surgical procedure.

In one embodiment, the ultrasound probe attached to the robot arm 16 may be automatically controlled by the system 524. For example, the phase and magnitude of the ultrasound transmitted signals from different elements of the ultrasound array may be programmed to electrically steer the beam in different directions, providing a computer controlled version of the manually swept ultrasound probe. The phase of the received signal has its phase similarly adjusted to create properly aligned image data. In this manner, the direction of the ultrasound beam may be electronically adjusted by the system 10 to guide the ultrasound probe to desired locations.

In another embodiment, the ultrasound patient tracking 520 includes a dynamic ultrasound probe 526. The dynamic ultrasound probe 526 mounts to the skin similarly to an ultrasound array, with a mechanical actuator that continuously sweeps back and forth within the limits of the adhesive patch, gently probing the same region of skin. The dynamic probe 526 may provide a two-dimensional visualized region from a simple linear probe, requiring less signal processing complexity than an ultrasound array. Because the probe 526 physically moves on the skin, it may also be less susceptible to setup errors that could happen when mounting a static ultrasound probe to the skin (e.g., air bubbles, regions in poor contact). That is, as the actuated probe 526 moves, it may dislodge air bubbles and settle into better contact with the skin. The dynamic ultrasound probe 526 continuously and systematically scans the targeted region, which enhances the probe's ability to capture detailed ultrasound images of the area.

In another embodiment, one arm 16 of the surgical robot 10 may be dedicated to the task of acting as a patient reference 528. The dedicated robot arm 16 may hold an optically tracked ultrasound probe and move as needed to position the probe over a trackable piece of bone near the surgical site. Robotic movement may include both positioning of the probe over regions of interest and sweeping back and forth to track areas of interest in two dimensions. Any slight movement of the patient may be sensed from the ultrasound tracking 528, and the position of the probe relative to the robot 10 may be sensed from joint encoders and/or optical tracking of the ultrasound probe itself. The robot 10 may also be able to reposition the arm 16 dynamically to provide optimal line of sight to the cameras 24 and to stay out of the way of the surgeon while continuously tracking patient position. Meanwhile, additional arms 16 of the robot 10 may perform surgery or other tasks, with relative positions of the robot arms 16 continuously measured, for example, from joint encoders. The dedicated ultrasound arm 528 may capture detailed ultrasound images for patient tracking.

In a variation on the embodiment where one arm 16 of the robot 10 holds a tracked ultrasound probe, the robot arm 16 may instead hold a non-tracked probe with known position relative to the robot 10 and then compute the position of the probe from forward kinematics and encoder readings on each joint of the robot 10 relative to an array mounted elsewhere. Doing so may prevent line of sight issues between the cameras 24 and the ultrasound probe from being obscured. The optical tracker on the robot 10 may be mounted elsewhere on the robot 10 where it has good line of sight, or the optical tracker may be held by another arm 16 of the robot 10, allowing that arm 16 to position itself optimally for line of sight. In a case where a first arm 16 holds the optical tracker and a second arm 16 holds the ultrasound probe, the position of the tracker relative to the robot base 12 may be computed from forward kinematics and the encoders on the first arm 16, and the position of the ultrasound relative to the base 16 may be computed from forward kinematics and the encoders on the second arm 16. With base 12 to first arm 16 and base 12 to second arm 16 transformations known, the transformation from optical tracker to ultrasound probe can be computed.

In another embodiment, a flexible multi-part sensor 530 is adhered to the patient, with ultrasound probes attached to various parts of the flexible sensor 530. The flexible sensor 530 stays in good contact with the patient because it conforms to the patient's unique anatomy. To account for the flexibility of the sensor 530 and provide accurate optical tracking of bone, the flexing of the sensor 530 may be continuously tracked by monitoring fiducials on the sensor's encasement. Sensor flexing data may be included to find the overall transformation of coordinates from anatomical coordinate system to optical tracking coordinate system.

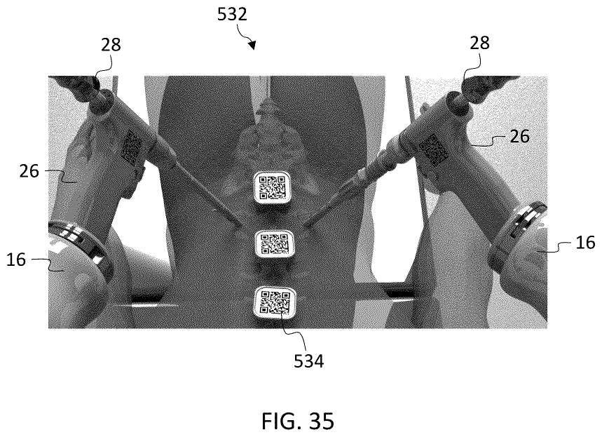

In another embodiment, multiple adhered and tracked ultrasound sensors 532 may be attached to the skin of the patient over different levels of the spine or other anatomy where, for example, a deformity correction procedure is being performed. Using ultrasound and optical tracking, the sensors 532 may non-invasively measure the positions of underlying bone relative to cameras and track correction of the deformity in real-time. The optical tracking on the sensors 532 may be low profile or based on sensor shape and visible light. Alternatively, as shown in FIG. 35, the sensors 532 may be marked with trackable fiducials such as quick-response (QR) codes 534. Multiple sensors 532 may improve the tracking robustness due to line of sight issues (e.g., if one sensor is blocked others are still tracking). Full 6-degree-of-freedom tracking may not be necessary in such an application where the movement of bones is being tracked if biomechanical modeling of the tissues is and the movement of each fiducial serves as a parameter entry in the biomechanical model to provide a high-certainty estimate of 3D movement. Using such an approach, surgical correction of scoliosis from T1-T12, for example, may be tracked on the operating table.

In another related embodiment, two or more ultrasound sensors 532 may be temporarily attached to the patient's back postoperatively to remain in place long-term, or may be briefly reattached in approximately the same place at a later time. Since the sensors 532 detect underlying bone, it is not important that they be reattached in exactly the same position. The sensor 532 may find the underlying bone surface and register it to the original preop CT. Then, at any time over the course of weeks, months, or years, the progress or regression of the correction may be monitored from the ultrasound and tracked sensor position. Thus, ultrasound sensors 532 may be used to monitor the patient's progress over time.

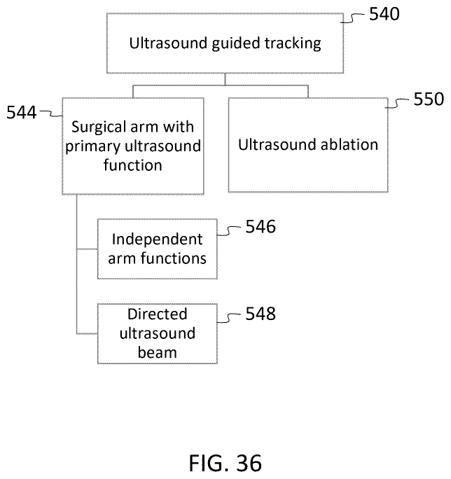

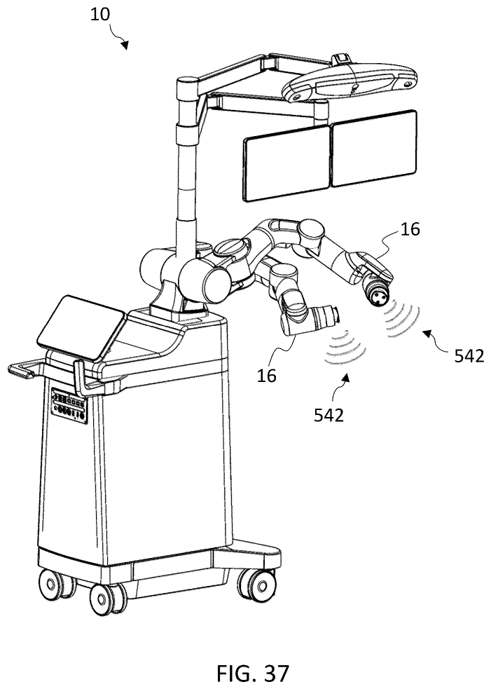

Turning now to FIG. 36, ultrasound guided tracking 540 may be used on one or both of the surgical arms 16 of the robotic system 10. As shown in FIG. 37, one or both of the surgical arms 16 may include an imaging probe 542. Imaging probes 542 may include ultrasound transducers, sonography probes, linear or convex array transducers, endocavity probes, or other suitable imaging systems. The imaging probes 542 may be integrated into the surgical arms 16, integrated into an end effector 26, or offered as a separate component, for example. Ultrasound guided tracking 540 may further include: (1) a dedicated surgical arm 16 with primary ultrasound function 544 while the other arm 16 performs surgical functions; (2) independent arm functions 546 that are coordinated based on ultrasound data; (3) a directed ultrasound beam 548 across a given path, for example, for focused cuts; and (4) ultrasound ablation devices 550 for focused ablation of tissues, for example, if ultrasound waves converge.

The robotic system 10 may be configured such that a dedicated surgical arm 16 is primarily for ultrasound function 544. For robotic systems with two or more surgical arms 16, it is contemplated that one arm's primary function may be to position the ultrasound probe 542 near the anatomy of interest, controlling the direction of the emitted ultrasound and helping to visualize what obstacles tools introduced by the other arm(s) 16 of the robot 10 may encounter as they penetrate. Optical tracking and ultrasound tracking may be used together to navigate a guide tube for receiving an instrument to a trajectory of interest. For example, if a lateral spinal interbody implant is being inserted through the tube held by one robotic arm 16, the second robotic arm 16 holding the ultrasound probe 542 may position its probe 542 over the skin and perpendicular to the trajectory of the first arm 16. The probe 542 may then sweep up and down along the trajectory, from entry down to the spine, visualizing anatomy such as nerves and blood vessels that may be encountered as the implant is inserted through the tube held by the first arm 16.

In a similar application, each surgical arm 16 performs independent arm functions 546. For example, one robot arm 16 holds the ultrasound probe 542 and directs it across the path of incision/drilling/sawing by another robot arm 16 or by a surgeon performing freehand surgery or surgery through a robot-guided tube. The ultrasound monitors progress of the resection and confirms whether the cuts are occurring in the planned region. In such an application, if the user attempts to cut outside the planned or allowable area, ultrasound 542 detects deviation and the robot arm 16 through which the cutting tool is being used can power down its blades or bits, apply course correction, or apply a counter force to prevent cutting outside of the allowable region. Such a process provides risk mitigation in case of, for example, discrepancy in the actual and programmed cutting blade size. Although performing independent functions, the surgical arms 16 may be coordinated and synchronized.

In a related embodiment, the ultrasound probe 542 provides a directed ultrasound beam 548. The ultrasound beam 548 is directed across the path of surgery, for example, left to right across the neck while a tool's trajectory is posterior to anterior, and monitors tool progress as a test cut is applied. The test cut may be programmed to be well within the dimensions of the final intended cut. Ultrasound 542 may then be used to measure the size and location of the test cut and to determine whether a final cut may be positioned and sized as intended. Based on the test cut, programming for the final cut may be updated and then the final cut may be performed. The test cut, evaluation, and adjustments may be performed manually or may be done automatically by the system 10. In one embodiment, the test cut may automatically be programmed at 50% inside the intended final cut, after which the analysis of cut position is performed and a dialog appears, prompting the user to accept or reject final cut modifications before then canceling or applying the final cut at 100% of intended size.

In another embodiment, two or more arms 16 of the robot 10 each hold focused ultrasound ablation devices 550. A focused ultrasound ablation device 550 may have a curved probe that is concave relative to the skin, directing ultrasound waves slightly toward each other, or may be electrically focused by adjusting the phase and magnitude of different ultrasound emitters on a flat surface. When any one focused ultrasound ablation device 550 emits ultrasound waves, the single wave does not damage tissues encountered. However, when two or more devices are positioned in such a way that the waves intersect, for example, where the waves converge at 90°, then the ultrasound waves combine at the focal point of their intersection with enough magnitude to focally ablate tissues such as tumors. The phases of the ultrasound waves may be set such that they have constructive interference at the point of interest. It is contemplated that the ultrasound waveform may be modified throughout the procedure, starting with a low energy waveform used for navigating the robotic arms, then switching to a high energy waveform and more focused waveform when ready to perform ablation. The focused ablation may precisely target tissues, while preserving nearby healthy tissues.

Although several embodiments of the invention have been disclosed in the foregoing specification, it is understood that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. It is further envisioned that features from one embodiment may be combined or used with the features from a different embodiment described herein. Moreover, although specific terms are employed herein, as well as in the claims which follow, they are used only in a generic and descriptive sense, and not for the purposes of limiting the described invention, nor the claims which follow. The entire disclosure of each patent and publication cited herein is incorporated by reference in its entirety, as if each such patent or publication were individually incorporated by reference herein. Various features and advantages of the invention are set forth in the following claims.

What is claimed is:

1. A surgical robotic system comprising:
a moveable base station, including an on-board computer;
a display electronically coupled to the computer;
a camera electronically coupled to the computer and configured to detect one or more tracking markers;
a surgical arm electronically coupled to the computer and movable based on commands processed by the computer;
an end effector attachable to the surgical arm; and
a sterile drape assembly attachable to the surgical arm, the sterile drape assembly including a sterile drape and a connector cap attached to the sterile drape, wherein the sterile drape assembly provides for power and data transmission from the surgical arm to the end effector.

2. The system of claim 1, wherein the connector cap houses an electrical connector, which transmits power and data from the surgical arm to the end effector.

3. The system of claim 1, wherein the surgical arm includes an end effector interface having a mounting flange with a conducive pad and a ferrous target.

4. The system of claim 3, wherein the end effector includes a clamp with a mounting flange, conducive pad, and ferrous target.

5. The system of claim 4, wherein the electrical connector of the connector cap includes a printed circuit board with pogo pins and a magnet on each side.

6. The system of claim 5, wherein the magnets on the connector cap allow for a magnetic connection with the ferrous targets on the surgical arm and end effector, respectively.

7. The system of claim 5, wherein a first set of pogo pins on the connector cap align with the conducive pad on the surgical arm and a second set of pogo pins on the connector cap align with the conducive pad on the end effector, thereby establishing electrical connectivity from the surgical arm to the end effector.

8. The system of claim 1, wherein the connector cap includes an embossed edge to facilitate proper alignment when attaching the connector cap to the surgical arm.

9. A multi-arm surgical robotic system comprising:
a moveable base station, including an on-board computer;
a display electronically coupled to the computer;
a camera electronically coupled to the computer and configured to detect one or more tracking markers;
a pair of surgical arms electronically coupled to the computer and movable based on commands processed by the computer; and
an ultrasound probe to register or track patient anatomy of a patient during a surgical procedure.

10. The system of claim 9, wherein an automatic ultrasound registration process is configured to be completed by the system.

11. The system of claim 10, wherein the automatic ultrasound registration includes a rough calibration by scanning the patient anatomy and a fine calibration for focused images.

12. The system of claim 10, wherein during the automatic ultrasound registration, frames of optical tracking data are synchronized with ultrasound data.

13. The system of claim 9, wherein the ultrasound probe is attached to one of the surgical arms and is automatically controlled by the system to intelligently guide the ultrasound imaging.

14. The system of claim 9, wherein the ultrasound probe includes a flexible multi-part ultrasound sensor configured to adhere to the patient.

15. The system of claim 9, wherein the ultrasound probe includes multiple ultrasound sensors configured to mount to the skin of the patient to track movement of the patient anatomy.

16. The system of claim 15, wherein the multiple ultrasound sensors include machine vision markings for optical tracking of the sensors.

17. A method by a surgical robot system comprising:
providing a multi-arm surgical robotic system comprising a pair of surgical arms, a display, and a camera supported on a single mobile cart, and an ultrasound probe, which is integrated into the surgical arm, integrated into an end effector, or offered as a separate component;
positioning the multi-arm surgical robotic system near an operating room table;
scanning a patient with the ultrasound probe to register or track patient anatomy; and
performing a surgical procedure with the assistance of the surgical arms.

18. The method of claim 17 further comprising performing a rough calibration by automatically scanning the patient with the ultrasound probe, and once anatomy is identified, replacing the rough calibration with a fine calibration to optimize imaging.

19. The method of claim 17, wherein one surgical arm is dedicated to holding the ultrasound probe, and positioning the ultrasound probe over regions of interest to track any movement from the patient, while the other surgical arm performs any surgical tasks.

20. The method of claim 17 further comprising attaching ultrasound sensors to the skin of the patient at a later time to track the patient's progress.
